(19) United States
(12) Patent Application Publication
(10) Pub. No.: US 2004/0006070 A1
(43) Pub. Date: Jan. 8, 2004

(54) Intraspinal continuous infusion of midazolam hydrochloride for the treatment of pain

(76) Inventors: Samuel J. Hassenbusch, Houston, TX (US); Mary Jane Johansen, Sugar Land, TX (US); Tamara Lee Gradert, Bastrop, TX (US); William C. Satterfield, Elgin, TX (US); Larry Trissel, Houston, TX (US)

Correspondence Address: FULBRIGHT & JAWORSKI L.L.P., 600 CONGRESS AVE., SUITE 2400, AUSTIN, TX, 78701, US

(21) Appl. No.: 10/375,911

(22) Filed: Feb. 27, 2003

Related U.S. Application Data
(60) Provisional application No. 60/359,866, filed on Feb. 27, 2002.

(51) Int. Cl. A61K 31/551

(52) U.S. Cl. 514/220

(57) ABSTRACT

The invention provides a preservative-free midazolam hydrochloride formulation that is less toxic, and more effective than present opioid therapies for alleviation of pain. Additionally, by an intrathecal infusion system for continuous administration of preservative-free midazolam hydrochloride the present invention circumvents breakthrough pain episodes often encountered with other means of opioid administration. The present invention further provides a novel method of treating pain that is of either non-neuropathic or neuropathic origin. Overall that present invention provides a method of treating cancer pain in patients by continuous intrathecal infusion of preservative-free midazolam hydrochloride.

The present invention claims priority to U.S. Provisional Patent Application Serial No: 60/359,866 filed on Feb. 27, 2002. The entire text of the above-referenced disclosure is specifically incorporated herein by reference. without disclaimer.

### BACKGROUND OF THE INVENTION

1. Field of the Invention

The present invention relates generally to the fields of pharmacology and pharmacotherapy. More particularly, it concerns methods for treating pain. In particular, the present invention relates to methods for treating pain by intraspinal administration of a benzodiazepine-GABAA receptor agonist, midazolam hydrochloride.

2. Description of Related Art

Many if not most ailments of the body cause pain. Generally, pain is experienced when the free nerve endings which constitute the pain receptors in the skin, as well as in certain internal tissues, are subjected to mechanical, thermal or chemical stimuli. The pain receptors transmit signals along afferent neurons into the central nervous system and then to the brain. Sometimes pain results when the nerve pathways themselves are injured. Pain is felt when the brain receives the signal from nerves to which damage is occurring. All types of pain are transmitted this way, including cancer pain.

The causes of pain can include inflammation, injury, disease or by treatments, muscle spasm and the onset of a neuropathic event or syndrome. Ineffectively treated pain can be devastating to the person experiencing it by limiting function, reducing mobility, complicating sleep, and dramatically interfering with the quality of life.

Pain caused by disease, or treatment thereof, is common in people with cancer, although not all people with cancer experience pain. Approximately 30% to 50% of people with cancer experience pain while undergoing treatment, and 70% to 90% of people with advanced cancer experience pain (Leasage and Portenoy, 1999).

Currently, pain that is mild to moderate is treated with nonsteroidal anti-inflammatory drugs (NSAIDS). However, if the pain is not relieved by NSAIDS alone, treatment with a fixed-dose combination containing codeine or oxycodone with aspirin or acetaminophen is implemented. If pain is not well-controlled at that dose level, use of a single entity opioid such as oxycodone is usually a further treatment option.

For pain that is moderate to severe, opioids (morphine, oxycodone, codeine, methadone, levorphanol, and fentanyl) are the major class of analgesics used because of their effectiveness, ease of titration, and favorable risk-to-benefit ratio. Morphine is the only such opioid designated by the World Health Organization as the preferred analgesic. Currently morphine is the only FDA approved analgesic for intrathecal therapy in treating pain. However, morphine as well as other opioids, are associated with significant side effects and are often ineffective at treating neuropathic pain.

For the treatment of pain, various studies have employed the use of midazolam hydrochloride in combination with morphine or other opioids. Animal studies using a sheep or dog model have also used bolus administration of midazolam. The efficacy and toxicity observed in these studies have been documented in the art (Serrao et al., 1990, 1992; Schoeffler et al., 1991; Madsen et al., 1990; Aguliar et al., 1994; Kyles et al., 1995). These studies utilized a midazolam preparation containing a preservative, and/or utilized bolus administration of the drug; and/or used the drug in combination with other analgesics such as the opioid morphine. A few of the animal studies have utilized a preservative-free midazolam hydrochloride for bolus administration.

### SUMMARY OF THE INVENTION

The present invention overcomes the deficiencies in the art by providing a novel approach to the treatment of pain of either non-neuropathic or neuropathic origin. Thus, in accordance with the present invention, there is provided a method for treating pain in a subject comprising intraspinal administration to said subject of an analgesic formulation comprising preservative-free midazolam, wherein said formulation is substantially free of other analgesic substances. In one embodiment, the treatment is for neuropathic pain or non-neuropathic pain. In a particular embodiment, high doses of midazolam are provided at the daily dose of at least about 1.0 mg. In another particular embodiment, high doses of midazolam are provided at the daily dose of at least about 5.0 mg. In yet another embodiment, doses of midazolam are provided at the daily dose of at least about 10.0 mg.

In still yet another embodiment, midazolam is provided at a daily dose of at least about 15.0 mg.

It is contemplated that the formulation of midazolam may be administered gradually over a time period of greater than one minute; greater than ten minutes; greater than thirty minutes; greater than sixty minutes; greater than one-hundred twenty minutes;

greater than four hours; greater than eight hours; greater than twelve eight hours; greater than twenty-four hours. It is further contemplated that the formulation of midazolam may be administered by a continuous infusion pump implanted subcutaneously in a subject having cancer.

In further embodiments, the subject may have cancer pain, again of a neuropathic or non-neuropathic origin. The subject may be opioid tolerant, or may suffer from opioid-resistant neuropathic pain. In still yet another embodiment, the subject is a human. In a further embodiment, the analgesic formulation of midazolam comprises at about 2.5 to about 5.0 mg/ml. In a particular embodiment, toxicity of preservative free midazolam is measured during treatment, and a dose modification is made based on the toxicity measurement. In further embodiments, pain relief is measured during treatment and dose modification is made based on the pain relief measurement.

In further embodiments of the present invention it is contemplated that the daily dose of midazolam hydrochloride is at least or at most 1, 2, 3, 4, 5, 6, 7, 8, 9, 10, 11, 12, 20 13, 14, 15, 16, 17, 18, 19, 20 mg.

In the context of the present invention, “cancer pain” is pain, which can be caused by the disease itself or by treatments that may be non-neuropathic, or neuropathic in origin.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 —Structure of midazolam hydrochloride.

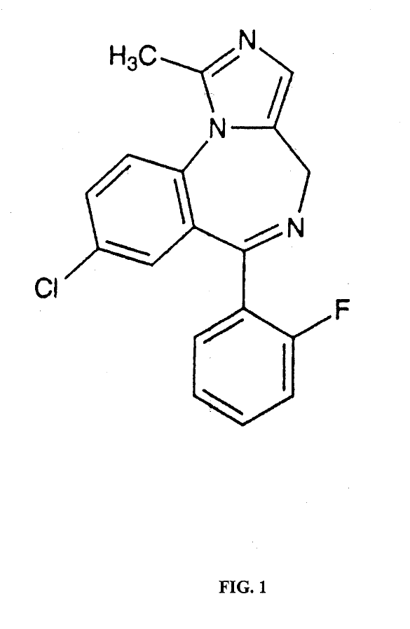

FIG. 2 —Continuous infusion system model in sheep.

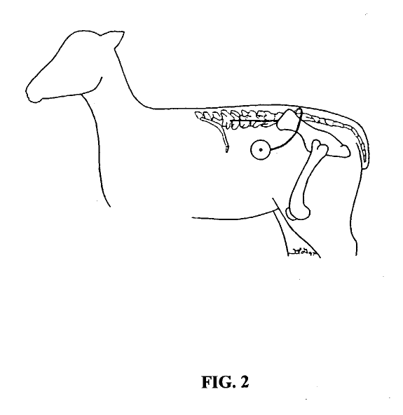

### DESCRIPTION OF ILLUSTRATIVE EMBODIMENTS

A. The Present Invention

The present invention concerns the use of a midazolam preparation that is preservative-free for the treatment of pain, such as non-neuropathic pain or neuropathic pain by intrathecal infusion. The invention further seeks to provide treatment for pain associated with or arising from a subject having cancer.

Since some subjects develop tolerance to the intraspinal infusion of opioids; and in other subjects the narcotic infusion produces side effects such as nausea, vomiting, sedation, and urinary retention; the present invention further contemplates an alternative method of treating pain to overcome these deficiencies. Tolerance of opioids such as morphine can develop to the point where intrathecal doses as high as 50 mg/day are ineffective in controlling pain. The present invention therefore seeks to provide an alternative for morphine, the only FDA approved intrathecal infusion treatment for pain that is both safe and less toxic.

The major advantages of the present invention as compared to current usage of midazolam are: (1) a midazolam preparation that is preservative-free and therefore less toxic than commonly used midazolam preparations containing preservatives such as benzyl alcohol; and (2) a continuous intraspinal infusion model for delivery of midazolam in the treatment of pain.

Another major advantage of the present invention is that intraspinal infusion of midazolam hydrochloride produces most, if not all, of its effects at the spinal levels rather than at the brainstem or peripheral nerve sites. Therefore, utilizing this spinal route of administration allows for delivery of midazolam hydrochloride in close proximity to target receptors resulting in higher local concentration of analgesics at their site of action, as well as providing pain relief that is often superior to that achieved when drugs are administered by other routes. Smaller doses can be delivered with minimal systemic exposure, thereby reducing the potential for side effects to develop. Additionally, an implantable infusion system allows for continuous infusion of drug to steady-state conditions, which will avoid breakthrough pain episodes often experienced with bolus administration.

B. Analgesia/Pain

The present invention seeks to overcome the deficiencies of current therapies in treating pain that is of a neuropathic or non-neuropathic origin by using preservative-free midazolam hydrochloride intraspinally/intrathecally. Pain can be divided into two broad categories: non-neuropathic (nociceptive) and neuropathic (non-nociceptive). These types of pain differ in their causes, symptoms, and responses to analgesics.

1. Non-Neuropathic Pain

Non-neuropathic (nociceptive or somatic) pain results from direct stimulation of intact afferent nerve endings and is characterized usually as dull, sharp or aching pain which is responsive to analgesics. Some examples of nociceptive pain include: bone pain (e.g., from a fracture, bone metastases, etc.); pain elicited by tissue injury; pressure pain; cancer pain. This type of pain can also be well controlled if the painful stimulus can be removed or treated with surgery, radiation therapy, or chemotherapy.

Non-neuropathic pain may also be acute or chronic or inflammatory. Acute pain usually starts suddenly, may be sharp, and often triggers visible bodily reactions such as sweating, elevated blood pressure, and more. Chronic pain lasts, and pain is considered chronic when it lasts beyond the normal time expected for an injury to heal or an illness to resolve. Inflammatory pain can occur when tissue is damaged, as can result from surgery or due to an adverse physical, chemical or thermal event or to infection by a biologic agent.

Although these types of non-neuropathic pain can be treated with current analgesics, there many drawbacks and deficiencies such as widespread systemic distribution of the drug, undesirable side effects, and short drug efficacy durations which necessitate frequent drug readministration with possible resulting drug resistance. The present invention therefore seeks to overcomes these drawbacks and deficiencies in treating non-neuropathic pain.

2. Neuropathic Pain

Other types of pain that may be treated by preservative-free midazolam hydrochloride include neuropathic pain. Neuropathic pain is a persistent or chronic pain syndrome that can result from damage to the nervous system, the peripheral nerves, the dorsal root ganglion or dorsal root, or to the central nervous system. This type of pain may exhibit opioid resistance or require higher opioid doses to achieve pain relief. Cancer pain is one such type of pain related to neuropathic pain and caused by tumor or treatment-related nerve damage, shingles, post-herpetic neuralgia, and phantom limb pain.

Current methods to treat neuropathic pain, such as by local anesthetic blocks targeted to trigger points, peripheral nerves, plexi, dorsal roots, and to the sympathetic nervous system have only short-lived anti-nociceptive effects. Additionally, longer lasting analgesic treatment methods, such as blocks by phenol injection or cryotherapy raise a considerable risk of irreversible functional impairment. Furthermore, chronic epidural or intrathecal (collectively “intraspinal”) administration of drugs such as clonidine, steroids, opioids or midazolam (containing preservative) have significant side effects and questionable efficacy. The present invention therefore provides an alternative to safely and effectively treat neuropathic pain, such as cancer pain, using preservative-free midazolam hydrochloride intraspinally/intrathecally.

3. Assessing/Rating Pain

Due to the multidimensional nature of pain, use of pain assessment tools provides more complete information on the nature of the pain and the effectiveness of pain treatments. Both qualitative and quantitative pain assessment are an important part of any study involving pain treatment. Qualitative description of the location, frequency and characteristics of the pain is important to assess pain type described above. As is known to those skilled in the art, the McGill Pain Questionnaire has been validated and found reliable in many studies including those involving cancer pain treatments (Graham et al., 1980; Kremer et al., 1882; Littman et al., 1985; Jensen et al., 1993).

Quantification of pain intensity can be assessed by asking the subject to rate the pain using numeric or visual scales at multiple intervals, tracking the pain over time, and with changes in therapy. The most reproducible and consistent methods are: (1) the visual analog scale (VAS), which uses a 10 cm horizontal measuring bar extending from no pain to worst pain, and (2) the verbal digital scale (VDS) which involves numerically rating the pain on scales of 0 to 10 or 0 to 100 (Melzack et al., 1975; Ahles et al., 1983; Merskey et al., 1986; Bonica et al., 1990). These types of pain assessment tools may be used in the present invention to determine the efficacy of preservative-free midazolam hydrochloride in treating subjects experiencing pain, i.e., cancer pain.

C. Intraspinal/Intrathecal Infusion

Current drug infusion methods for the treatment of pain relate primarily. to morphine, the only approved FDA analgesic for treating pain intrathecally. The present invention applies particularly to intrathecal drug infusion of preservative-free midazolam hydrochloride in the treatment of cancer pain. In the treatment of some types of pain such as in cancer pain, more invasive alternative treatments may be required to achieve pain control other than systemically-administered opioid analgesics.

Therapeutic administration of certain drugs intraspinally, that is to either the epidural space or to the intrathecal space, is known to those skilled in the art. Administration of a drug directly to the intrathecal space can be by either spinal tap injection or by catheterization. Intrathecal drug administration can avoid the inactivation of some drugs when taken orally as well and the systemic effects of oral or intravenous administration. Additionally, intrathecal administration permits use of an effective dose which is only a fraction of the effective dose required by oral or parenteral administration. Furthermore, the intrathecal space is generally wide enough to accommodate a small catheter, thereby enabling chronic drug delivery systems. Moreover, it is known to one skilled in the art, to treat pain by intraspinal administration of the opioids morphine and fentanyl (Gianno et al., 1996).

1. Intraspinal Midazolam Therapy

Midazolam Infusion.

The pump is filled with 18 mL (capacity) midazolam hydrochloride (2.5 or 5.0 mg/ml). The dead space within the pump and catheter tubing is then primed with 400 μl the midazolam solution (pump and tubing dead space is 360 to 380 μl ). The dosing rate begins at 1 mg/day, and is escalated as described below.

Dose Escalation.

Doses as contemplated with the present invention are chosen based on the prior art. Sedation and somnolence have been the toxicities reported with 1-5 mg/day. Starting on Day 1 of the study, the pump is programmed for an increasing infusion rate. Intrasubject dose escalation is performed every 2 weeks over an 8 week period according to the following schema: Pump Rate for Course 1: week 1: 1 mg/day; week 3: 2 mg/day; week 5: 3 mg/day; week 7: 4 mg/day; week 9: 5 mg/day; week 11: Pain response is qualitatively and quantitatively assessed as previously described.

D. Route of Administration

1. The Pump Implantation Procedure

Using the intraspinal route of administration, effective analgesics such as preservative-free midazolam hydrochloride can exert their activity at sites in the spinal cord, with limited exposure to brainstem and midbrain levels, and essentially no exposure to supratentorial brain structures. Because of this localization to the effector site, spinally administered analgesics can be given at lower doses, thereby also minimizing systemic exposure and offering relief from pain.

Implanted spinal infusion pumps, and programmable pumps (pumps with infusion rates that can be changed through the skin via radiotelemetry) are well known to those skilled in the art. Of these, the most common is the SynchroMede infusion pump (Medtronic, Inc., Minneapolis, Minn.), used in the present invention. Studies at multiple institutions have demonstrated the reliability of this pump for drug infusion, with a device-related complication rate of approximately 6% and a rate of overinfusion of 1.4%. A low infection rate of 2% shown with use of this pump and spinal catheter system also demonstrates its safety for use in cancer subjects. The pump has the ability to infuse at rates of 0.002-0.90 ml/hr with a reservoir volume of 18 ml. The sideport at the edge of the pump allows aspiration of fluid in the catheter as well as cerebrospinal fluid for flushing of the catheter. The location chosen for the spinal catheter tip is dependent on the length of the spinal catheter and safety with regard to avoidance of spinal cord damage.

Implanting an intrathecal catheter and pump is a surgical procedure that takes 1-2 hours to complete. The pump itself is about the size of a hockey puck allows for the infusion of analgesic substances such as preservative-free midazolam hydrochloride into the cerebral spinal fluid. Infusion is usually accomplished with a thin catheter implanted in the spinal canal and connected to a pump which resides under the skin in the abdomen. The placement of the spinal catheter is performed with a puncture at the L1-2 or L2-3 level of the spinal cord, with passage of the catheter tip between the T7 to T11 level. The pump is then placed in the subcutaneous fat of the abdomen, just below the ribs. A tube connecting the pump and the intrathecal catheter goes around the flank.

In the present invention, the pump delivers very small doses of a substance (i.e., preservative-free midazolam hydrochloride) into the spinal fluid. Because of the direct nature of delivery of this substance, much lower doses are required to achieve good pain relief than required with oral medications. In addition, side effects of oral or systemic medications are seen far less frequently with intrathecal infusion. The pump is filled at the time of surgery and a low dose of narcotic is begun after surgery. The pump is easily refilled with little discomfort to the subject, and dose changes can be made with a special radiofrequency transmitter placed over the skin. The implanted pump can be programmed for continuous or intermittent infusion of the drug through the intrathecally located catheter. In the present invention, the pump is programmed for continuous infusion of preservative-free midazolam hydrochloride.

Before infusing midazolam through a permanently implanted intrathecal pump, subjects undergo infusion of a narcotic (usually morphine) into their spinal canal in order to see whether they obtain benefit opioids given by this route of administration. In addition, possible side-effects with intrathecal narcotics can be judged. If subjects do not obtain adequate pain relief or experience intolerable side effects with opioid intraspinal infusion, the intraspinal opioid dose is converted to a systemic opioid dose and midazolam intraspinal therapy provided.

2. Test Infusion of Opioids

a. Calculation of Systemic Equivalent Doses of Intraspinal Opioid.

The opioid dose being administered intraspinally is first converted to systemic morphine equivalents. This conversion is based on approximate equipotent doses of opioids for different routes of delivery (e.g., intrathecal, epidural, systemic, oral) according to the following:

| | IV | IT | PO |
|---|---|---|---|
| Oxycodone | 200 | 10 | 600 |
| Hydromorphone | 33 | 1.7 | 100 |
| Morphine | 200 | 10 | 600 |

These formulas are based upon published studies of analgesic potency, and experience of the principal investigator with epidural and intrathecal infusions of these agents. Intrathecal infusion of morphine sulfate or hydromorphone has been found to be approximately 20 times more potent than intravenous infusion. Intrathecal infusion of hydromorphone has been found to be approximately 6 times more potent than intrathecal infusion of morphine sulfate. Oral doses may be increased from systemic equivalents by a factor of 3 to account approximately for different absorption rates between systemic and oral delivery.

b. Conversion of Opioid Spinal Infusion to Systemic Infusion or Oral Therapy.

The spinal infusion of opioid (morphine sulfate or hydromorphone) may be tapered off over one week prior to the initiation of midazolam therapy, and systemic dosing begun. Oral therapy is the preferred route for conversion from the spinal opioid infusion. The basal morphine dose is adjusted daily for pain relief during the week prior to midazolam initiation, until a stable morphine dose for pain relief is achieved. Two days prior to the start of midazolam therapy, the spinal infusion tubing is then flushed with saline by running the pump at 30 μL/hr for 48 hr.

The maximum allowed for each individual dose of rescue medication will be 15% of the total daily narcotic dose administered systemically (in morphine systemic equivalents). Rescue dose frequency will follow a schedule appropriate for the route of delivery (e.g., hourly for intravenous delivery, 4 hr for oral).

E. Combination Treatment/Therapies

In the present invention although preservative-free midazolam hydrochloride as a single agent is the preferred method of intraspinal/intrathecal infusion in treating cancer pain, it is further contemplated that other agents known in the art for treating pain may be combined with the present invention to further alleviate pain. In order to increase the effectiveness of a given therapy, it may be desirable to combine various compositions of analgesics with the preservative-free midazolam preparation of the present invention. It is further contemplated that, non-opioids, a surgical therapeutic agent (e.g., a surgical procedure) or a combination thereof, may be combined with preservative-free midazolam hydrochloride for intraspinal/intrathecal infusion in the treatment of pain.

Cancer pain can often be relieved by treatment with chemotherapy, hormonal therapy, surgery, radiotherapy, nerve blocks, psychological techniques, or a combination of these. However, the mainstay of chronic cancer pain management is opioid therapy. Drugs used to treat cancer pain include non-opioids, opioids, and adjuvant drugs.

The treatment of pain may employ a multifaceted approach of various medications and strategies such as: (a) nonsteroidal anti-inflammatory drugs, (b) antidepressants, (c) oral anti-arrhythmic medications (e.g., mexilitine hydrochloride if an intravenous infusion of xylocaine provides temporary relief), (d) adrenergic blocking compounds (e.g., propranolol hydrochloride, phentolamine), (e) calcium channel blocking agents, (f) anticonvulsants, and (g) aggressive physical and occupational therapy. In addition to these medications, sympathetic blocks and/or denervations, transcutaneous electrical nerve stimulation (Bonica, 1990; Hassenbusch et al., 1990 Nishiyama et al., 1999), intravenous phentolamine infusions, and regional (Bier-Block) guanethidine injections also have been utilized (Kyles et al., 1995; Valentine et al., 1996).

Therefore, the present invention contemplates the use of intraspinal/intrathecal infusion of preservative-free midazolam hydrochloride in combination with other modalities.

1. Non-Opioids

Non-opioids such as aspirin, or a nonsteroidal anti-inflammatory drug (NSAID) are effective for the treatment of mild pain. NSAIDs are preferred for the pain of bone metastases. The non-opioids all have an analgesic ceiling, that is, above a certain dose no further analgesic activity is to be expected. These non-opioids may be given in combination with preservative-free midazolam hydrochloride in the present invention to further alleviate cancer pain.

2. Calcium Channel Blockers/Antagonist

These aid in blocking the influx of calcium into cells. Ziconotide is the preferred calcium channel blocker in the treatment of pain. Other examples of a calcium channel blocker, that may be used with the present invention include: an arylalkylamine (e.g., bepridile, diltiazem, fendiline, gallopamil, prenylamine, terodiline, verapamil); a dihydropyridine derivative (felodipine, isradipine, nicardipine, nifedipine, nimodipine, nisoldipine, nitrendipine); a piperazine derivative (e.g., cinnarizine, flunarizine, lidoflazine); or a miscellaneous calcium channel blocker such as bencyclane, etafenone, magnesium, mibefradil or perhexiline. In certain embodiments a calcium channel blocker comprises a long-acting dihydropyridine (nifedipine-type) calcium antagonist.

3. Adjuvant Drugs

Other drugs which include corticosteroids, anticonvulsants, antidepressants, local anesthetics, and stimulants, may be given in combination with midazolam hydrochloride in the present invention. This is done to increase the effectiveness of the pain medication, treat symptoms, and relieve specific types of pain. Anti-depressant or anti-convulsant medications are used to treat neuropathic pain

4. Alpha-Adrenergic Agonists

Activation of these receptors have been shown to have antinociceptive properties. Epidural clonidine has been used in the treatment of chronic pain in humans. It is usually administered as an adjunct agent because of possible significant adverse cardiovascular effects, including bradycardia and hypotension.

5. Sodium Channel Agonists

Another route to pain relief is by opening sodium channels. Local anesthetics work via this mechanism. Bupivacaine is most commonly used. Local anesthetics are limited by the nature of their nonspecific blockade. Potential serious side effects are periods of orthostatic hypotension and bradyapnea. Other examples sodium channel agonists include lidocaine (xylocaine), tocainide (tonocard) and mexiletine (mexitil).

6. Radiation Therapy

Local or whole-body radiation therapy may increase the effectiveness of pain medication and other noninvasive therapies by directly affecting the cause of the pain (for example, by reducing tumor size).

7. Surgery

Surgery may be used to remove part or all of a tumor to reduce pain directly, relieve symptoms of obstruction or compression, and improve outcome, even increasing long-term survival.

8. Nerve Blocks

A nerve block is the injection of either a local anesthetic or a drug that inactivates nerves to control otherwise uncontrollable pain. Nerve blocks can be used to determine the source of pain, to treat painful conditions that respond to nerve blocks, to predict how the pain will respond to long-term treatments, and to prevent pain following procedures.

9. Neurologic Interventions

Surgery can be performed to implant devices that deliver drugs or electrically stimulate the nerves. In rare cases, surgery may be done to destroy a nerve or nerves that are part of the pain pathway.

F. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Toxicity and Nociceptive Testing of Midazolam in the Acute Pain Sheep Model-Open-Label (Known dose) Trial

The inventors have completed a toxicity and efficacy study of intrathecal midazolam hydrochloride in 15 sheep instrumented with Medtronic SynchroMed® infusion systems. The surgical procedure and the hardware used in these animals was analogous to that utilized in humans. The first sheep was tested for toxicity only (3 mg/day), and all subsequent animals tested for both toxicity and analgesic activity.

Open-label (known dose) trial: A trial with known midazolam doses was performed in the first 7 sheep. Animals were administered 3 mg/day (N=1), 5 mg/day (N=1), 10 mg/day (N=1), and 15 mg/day (N=4) for 43 days. These doses were chosen based upon previous intrathecal bolus dose studies in rats and humans, with conversion of from species to species based on cerebral spinal fluid (CSF) production rate and total CSF volume. Analgesic activity was assessed using a mechanical stimulus device which produces a stimulus of acute pain by application of force via a blunt needle applied to the shaved front foreleg of the animal. Force is applied with increasing pressure until the animal lifts its leg in response to the painful stimulus. To evaluate the analgesic effect of midazolam, response latencies were expressed as a percentage of the maximum possible effect, % MPE. The response latency is defined as follows:

$$\% MPE = \frac{Postdrug response \text{-} predrug response}{Cutoff \text{-} predrug response} \times 100$$

All treated animals exhibited significant pain relief. On most treatment days, sheep receiving midazolam had an increase in pain tolerance equivalent to 30 to 100% of maximal possible effect. The continuous infusion of midazolam in the open label sheep did not produce any behavioral, toxicological, or histopathologic changes related to the midazolam infusion in any subject.

Midazolam Sheep Nociceptive Testing and Physiological Data
Midazolam Sheep #518 Sandbur Slim (Sandy), Dose 5 mg/day

| | | | | | | | | Body |
|---|---|---|---|---|---|---|---|---|
| | | | | | | | Pulse | Temp. |
| | Mechanical | Allodynia | | Gait | Blood | | Rate | Normal |
| | Stimulus | Warm | Cold | Normal | pressure | | Normal | 101.3- |
| Day | % MPE | 45° C. | 6° C. | 0 | Sys | Dia | 55-115 | 104.0 |
| Baseline | — | 28 | 11 | 0 | 190 | 71 | 140 | 102.2 |
| Day 1 | 11.12 | 16 | 7 | 0 | 185 | 90 | 140 | 101.8 |
| Day 3 | 63.02 | 7 | 0 | 0 | 180 | 103 | 84 | 102.0 |
| Day 7 | 81.37 | 2 | 0 | 0 | 150 | 53 | 89 | 101.0 |
| Day 15 | 89.43 | 4 | 2 | 0 | 141 | 66 | 68 | 102.0 |
| Day 22 | 92.68 | 4 | 6 | 0 | 152 | 38 | 94 | 101.8 |
| Day 29 | 100 | 0 | 1 | 0 | 147 | 48 | 90 | 101.6 |
| Day 36 | 100 | 4 | 6 | 0 | 169 | 65 | 72 | 102.8 |
| Day 43 | 100 | 1 | 4 | 0 | 164 | 79 | 99 | 102.0 |

No clinical symptoms

Open Label Sheep
Midazolam Sheep #498 Toad, Dose 10 mg/day

| | | | | | | | | Body |
|---|---|---|---|---|---|---|---|---|
| | | | | | | | Pulse | Temp. |
| | Mechanical | Allodynia | | Gait | Blood | | Rate | Normal |
| | Stimulus | Warm | Cold | Normal | pressure | | Normal | 101.3- |
| Day | % MPE | 45° C. | 6° C. | 0 | Sys | Dia | 55-115 | 104.0 |
| Baseline | — | 77 | 17 | 0 | 147 | 81 | 103 | 103.2 |
| Day 1 | 100 | 3 | 1 | 0 | 177 | 98 | 98 | 102.0 |
| Day 3 | 68.48 | 10 | 8 | 0 | 143 | 73 | 79 | 104.2 |
| Day 7 | 17.95 | 19 | 2 | 0 | 136 | 66 | 95 | 102.8 |
| Day 15 | 75.91 | 2 | 6 | 0 | 156 | 109 | 77 | 102.4 |
| Day 22 | 43.92 | 0 | 2 | 0 | 153 | 71 | 104 | 101.8 |
| Day 29 | 100 | 11 | 8 | 0 | 118 | 66 | 67 | 101.8 |
| Day 36 | 91.74 | 2 | 5 | 0 | 153 | 59 | 89 | 103.0 |
| Day 43 | 62.1 | 2 | 3 | 0 | 149 | 58 | 78 | 103.2 |

No clinical symptoms

Open Label Sheep
Midazolam Sheep #457 Tazz, Dose 15 mg/day

| | | | | | | | | Body |
|---|---|---|---|---|---|---|---|---|
| | | | | | | | Pulse | Temp. |
| | Mechanical | Allodynia | | Gait | Blood | | Rate | Normal |
| | Stimulus | Warm | Cold | Normal | pressure | | Normal | 101.3- |
| Day | % MPE | 45° C. | 6° C. | 0 | sys | Dia | 55-115 | 104.0 |
| Baseline | — | 37 | 4 | 0 | 161 | 75 | 86 | 102.0 |
| Day 1 | −20.1 | 76 | 3 | 0 | 151 | 83 | 138 | 100.8 |
| Day 7 | 55.90 | 0 | 4 | 0 | 154 | 71 | 77 | 102.2 |
| Day 15 | 100 | 3 | 4 | 0 | 145 | 68 | 74 | 103.0 |
| Day 22 | 80.56 | 3 | 3 | 0 | 151 | 70 | 76 | 102.2 |
| Day 29 | 72.67 | 2 | 1 | 0 | 154 | 73 | 79 | 102.0 |
| Day 36 | 40.7 | 4 | 1 | 0 | 147 | 88 | 82 | 100.8 |
| Day 43 | 77.99 | 0 | 0 | 0 | 135 | 73 | 74 | 102.2 |

No clinical symptoms

Open Label Sheep
Sheep: Wacko #83
Midazolam dose: 15 mg/day

| | | | | | | | | Body |
|---|---|---|---|---|---|---|---|---|
| | | | | | | | Pulse | Temp. |
| | Mechanical | Allodynia | | Gait | Blood | | Rate | Normal |
| | Stimulus | Warm | Cold | Normal | pressure | | Normal | 101.3- |
| Day | % MPE | 45° C. | 6° C. | 0 | Sys | Dia | 55-115 | 104.0 |
| Baseline | — | 9 | 64 | 0 | 127 | 48 | 133 | 102.2 |
| Day 1 | 33.39 | 1 | 1 | 0 | 148 | 60 | 104 | 102.0 |
| Day 3 | −106.61 | 7 | 2 | 0 | 166 | 45 | 98 | 102.4 |
| Day 7 | 54.46 | 11 | 3 | 0 | 130 | 47 | 91 | 102.6 |
| Day 15 | 75.71 | 0 | 1 | 0 | 106 | 42 | 98 | 102.4 |
| Day 22 | 100 | 4 | 6 | 0 | 102 | 51 | 92 | 102.6 |
| Day 29 | −4.82 | 0 | 3 | 0 | 132 | 66 | 99 | 102.0 |
| Day 36 | −61.25 | 1 | 2 | 0 | 163 | 73 | 84 | 102.6 |
| Day 43 | 40.89 | 0 | 0 | 0 | 128 | 70 | 75 | 102.6 |

Necropsy: catheter placement confirmed as intrathecal, no gross lesions noted. Histology: mild catheter reaction.

Open Label Sheep
Sheep: Yacko #91
Midazolam dose: 15 mg/day

| | | | | | | | Pulse | Temp. |
|---|---|---|---|---|---|---|---|---|
| | Mechanical | Allodynia | | Gait | Blood | | Rate | Normal |
| | Stimulus | Warm | Cold | Normal | pressure | | Normal | 101.3- |
| Day | % MPE | 45° C. | 6° C. | 0 | Sys | Dia | 55-115 | 104.0 |
| Baseline | — | 2 | 43 | 0 | 144 | 61 | 147 | 101.6 |
| Day 1 | 10.18 | 9 | 15 | 0 | 138 | 65 | 115 | 102.8 |
| Day 3 | 49.16 | 5 | 9 | 0 | 143 | 55 | 80 | 102.8 |
| Day 7 | 21.31 | 12 | 9 | 0 | 156 | 56 | 84 | 102.6 |
| Day 15 | 100 | 5 | 1 | 0 | 110 | 44 | 86 | 103.0 |
| Day 22 | 78.04 | 2 | 0 | 0 | 156 | 64 | 72 | 102.0 |
| Day 29 | 100 | 3 | 1 | 0 | 151 | 32 | 82 | 104.4* |
| Day 36 | 41.57 | 1 | 2 | 0 | 160 | 66 | 66 | 103.0 |
| Day 43 | 58.36 | 7 | 9 | 0 | 149 | 60 | 69 | 102.2 |

*Temperature taken after nociceptive testing.
Necropsy: catheter placement confirmed as intrathecal, no gross lesions noted. Histology: mild catheter reaction.

Open Label Sheep
Sheep: Dot #85
Midazolam dose: 15 mg/day

| | | | | | | | | Body |
|---|---|---|---|---|---|---|---|---|
| | | | | | | | Pulse | Temp. |
| | Mechanical | ALLODYNIA | | GAIT | Blood | | Rate | Normal |
| | Stimulus | Warm | Cold | Normal | pressure | | Normal | 101.3- |
| Day | % MPE | 45° C. | 6° C. | 0 | Sys | Dia | 55-115 | 104.0 |
| Baseline | — | 4 | 39 | 0 | 127 | 75 | 83 | 102.2 |
| Day 1 | 58.39 | 1 | 14 | 0 | 128 | 68 | 92 | 103.4 |
| Day 3 | 27.67 | 2 | 2 | 0 | 109 | 49 | 89 | 103.0 |
| Day 7 | 70.92 | 2 | 9 | 0 | 110 | 67 | 86 | 103.0 |
| Day 15 | 92.59 | 3 | 2 | 0 | 133 | 59 | 117 | 102.6 |
| Day 22 | 83.88 | 0 | 0 | 0 | 130 | 53 | 85 | 102.8 |
| Day 29 | 68.63 | 3 | 3 | 0 | 128 | 77 | 72 | 104.6* |
| Day 36 | 66.56 | 5 | 5 | 0 | 120 | 57 | 66 | 103.0 |
| Day 43 | 100 | 2 | 2 | 0 | 155 | 69 | 71 | 103.2 |

*Temperature taken after nociceptive testing.
Necropsy: catheter placement confirmed as intrathecal, no gross lesions noted. Histology: mild catheter reaction.

Open Label Sheep
Midazolam Sheep: Bloodwork
Midazolam Sheep #473, Red Dose: 3 mg/day
There was not any pain testing done on this animal
Hematology

| | | | | | | | | Platlet | | | | | |
|---|---|---|---|---|---|---|---|---|---|---|---|---|---|
| | | | | | | | | Count | | | | | |
| | | | | | | | | ×10³/ | Differential Count: | | | | |
| | WBC | RBC | Hgb | Hct | MCV | MCH | MCHC | μl | q Absolute Values % | | | | |
| | ×10³/μl | ×10⁶/μl | g/dl | % | fl | Pg | G/dl | 250- | Mono | Lymph | Seg | Eosin | Atyp |
| Day | 4-12 | 9-15 | 9-15 | 27-45 | 28-40 | 8-12 | 31-34 | 750 | 0-6 | 40-75 | 10-50 | 0-10 | lym |
| Baseline | 6.2 | 11.62 | 12.1 | 38.2 | 30.1 | 9.5 | 31.7 | 573 | | 31 | 66 | 3 | |
| Pre- | | | | | | | | | | | | | |
| surgery | | | | | | | | | | | | | |
| Day 1 | 5.8 | 11.0 | 11.3 | 33.0 | 30.0 | 9.7 | 32.2 | 666 | 1 | 39 | 59 | 1 | |
| Day 15 | 5.1 | 12.50 | 12.9 | 41.0 | 29.6 | 9.3 | 31.5 | 336 | 1 | 53 | 40 | 6 | |
| Day 43 | 3.4 | 13.73 | 12.9 | 41.5 | 30.2 | 9.4 | 31.1 | 263 | 2 | 60 | 36 | 2 | |

Chemistries

| | Total | | | Alk | Crea- | Glu- | | Total | Albu- | | Gamma | | | | | |
|---|---|---|---|---|---|---|---|---|---|---|---|---|---|---|---|---|
| | Bili | | | Phos | tinine | cose | BUN | Prot | min | Choles- | GT | Phos | | Cl− | Na+ | K+ |
| | mg/dl | ALT | AST | U/L | mg/dl | mg/dl | mg/dl | g/dl | g/dl | terol | U/L | mg/dl | CPK | mEq/l | mEq/l | mEq/l |
| Day | 0.14-0.32 | U/L | U/L | 68-387 | 1-2.7 | 42-76 | 8-20 | 6-7.9 | 2.4-3 | mg/dl | 25-59 | 5-7.3 | U/L | 95-103 | 139-152 | 3.9-5.4 |
| Baseline | 0.2 | 21 | 278 | 48 | 1.2 | 121 | 11 | 6.3 | 3.4 | 77 | 72 | 6.7 | 544 | 105 | 151 | 4.4 |
| Day 1 | 0.2 | 22 | 239 | 41 | 1.0 | 91 | 10 | 6.5 | 3.4 | 72 | 69 | 6.9 | 392 | 111 | 153 | 4.4 |
| Day 15 | 0.1 | 1 | 85 | 59 | 1.2 | 74 | 10 | 6.7 | 3.7 | 79 | 59 | 5.8 | 98 | 114 | 152 | 4.6 |
| Day 43 | 0.0 | 8 | 61 | 73 | 1.5 | 59 | 15 | 6.4 | 3.4 | 78 | 58 | 5.3 | 82 | 108 | 150 | 5.6 |

Open Label Sheep
Midazolam Sheep #518, Sandbur Slim (Sandy), dose 5 mg/day
Hematology

| | | | | | | | | Platlet | | | | | |
|---|---|---|---|---|---|---|---|---|---|---|---|---|---|
| | | | | | | | | Count | | | | | |
| | | | | | | | | ×10³/ | Differential Count: | | | | |
| | WBC | RBC | Hgb | Hct | MCV | MCH | MCHC | μl | q Absolute Values % | | | | |
| | ×10³/μl | ×10⁶/μl | g/dl | % | Fl | pg | G/dl | 250- | Mono | Lymph | Seg | Eosin | Atyp |
| Day | 4-12 | 9-15 | 9-15 | 27-45 | 28-40 | 8-12 | 31-34 | 750 | 0-6 | 40-75 | 10-50 | 0-10 | lym |
| Baseline | 5.1 | 14.2 | 14.3 | 43.0 | 30.3 | 8.9 | 29.4 | 393 | 4 | 49 | 40 | 7 | |
| Day 1 | 8.4 | 11.44 | 12.2 | 35.0 | 30.6 | 9.6 | 31.4 | 642 | | 29 | 70 | 1 | |
| Day 3 | 6.4 | 10.75 | 11.3 | 33.0 | 30.7 | 9.3 | 30.1 | 546 | | 47 | 51 | 1 | 1 |
| Day 15 | 5.3 | 12.08 | 11.6 | 33.0 | 30.7 | 9.6 | 31.3 | 533 | | 62 | 33 | 5 | |
| Day 43 | 4.8 | 12.54 | 13.7 | 44.6 | 31.1 | 9.6 | 30.7 | 448 | | 48 | 47 | 4 | 1 |

Chemistries

| | Total | | | Alk | Crea- | Glu- | | Total | Albu- | | Gamma | | | | | |
|---|---|---|---|---|---|---|---|---|---|---|---|---|---|---|---|---|
| | Bili | | | Phos | tinine | cose | BUN | Prot | min | Choles- | GT | Phos | | Cl− | Na+ | K+ |
| | mg/dl | ALT | AST | U/L | mg/dl | mg/dl | mg/dl | G/dl | G/dl | terol | U/L | mg/dl | CPK | mEq/l | mEq/l | mEq/l |
| Day | 0.14-0.32 | U/L | U/L | 68-387 | 1-2.7 | 42-76 | 8-20 | 6-7.9 | 2.4-3 | mg/dl | 25-59 | 5-7.3 | U/L | 95-103 | 139-152 | 3.9-5.4 |
| Baseline | 0.3 | 31.0 | 185 | 105 | 1.2 | 95 | 14 | 7.1 | 3.7 | 89 | 86 | 7.7 | 473 | 110 | 158 | 4.7 |
| Day 1 | 0.2 | 12.0 | 112 | 157 | 1.1 | 8.8 | 15 | 7.0 | 3.6 | 96 | 79 | 7.2 | 148 | 108 | 154 | 4.9 |
| Day 3 | 0.1 | 10.0 | 99 | 99 | 1.4 | 122 | 5 | 6.6 | 3.8 | 87 | 79 | 5.7 | 834 | 113 | 151 | 4.4 |
| Day 15 | 0.1 | 13.0 | 74 | 100 | 1.6 | 74 | 13 | 6.7 | 4.1 | 83 | 90 | 5.1 | 172 | 112 | 151 | 4.6 |
| Day 43 | 0.1 | 12.0 | 76 | 89 | 1.7 | 89 | 7 | 6.2 | 3.5 | 69 | 68 | 6.4 | 787 | 111 | 152 | 4.6 |

Open Label Sheep
Midazolam Sheep #498, Toad, Dose 10 mg/day
Hematology

| | | | | | | | | Platlet | | | | | | | |
|---|---|---|---|---|---|---|---|---|---|---|---|---|---|---|---|
| | | | | | | | | Count | | | | | | | |
| | | | | | | | | ×10³/ | Differential Count: | | | | | | |
| | WBC | RBC | Hgb | Hct | MCV | MCH | MCHC | μl | q Absolute Values % | | | | | | |
| | ×10³/μl | ×10⁶/μl | g/dl | % | fl | pg | g/dl | 250- | | Mono | Lymph | Seg | Eosin | Atyp | Baso |
| Day | 4-12 | 9-15 | 9-15 | 27-45 | 28-40 | 8-12 | 31-34 | 750 | Bands | 0-6 | 40-75 | 10-50 | 0-10 | lym | 0-3 |
| Baseline | 5.4 | 11.29 | 12.0 | 35.0 | 31.0 | 9.1 | 9.5 | 325 | | 2 | 52 | 31 | 15 | | |
| Day 1 | 6.0 | 11.53 | 11.1 | 31.0 | 30.1 | 9.6 | 32.0 | 592 | | 1 | 28 | 68 | | 2 | 1 |
| Day 15 | 8.6 | 14.39 | 13.3 | 44.8 | 31.1 | 9.2 | 29.7 | 42 | 8 | 3 | 66 | 23 | | | |
| Day 43 | 9.7 | 10.03 | 10.5 | 34.7 | 30.9 | 9.3 | 30.3 | Ade- | | 1 | 63 | 33 | 3 | | |
| | | | | | | | | quate | | | | | | | |

Chemistries

| | Total | | | Alk | Crea- | Glu- | BUN | Total | Albu- | | Gamma | Phos | | | | |
|---|---|---|---|---|---|---|---|---|---|---|---|---|---|---|---|---|
| | Bili | | | Phos | tinine | Cose | Mg/ | Prot | min | Choles- | GT | Mg/ | | Cl− | Na+ | K+ |
| | mg/dl | ALT | AST | U/L | mg/dl | mg/dl | dl | g/dl | g/dl | terol | U/L | dl | CPK | mEq/l | mEq/l | mEq/l |
| Day | 0.14-0.32 | U/L | U/L | 68-387 | 1-2.7 | 42-76 | 8-20 | 6-7.9 | 2.4-3 | Mg/dl | 25-59 | 5-7.3 | U/L | 95-103 | 139-152 | 3.9-5.4 |
| Baseline | 0.1 | 5.0 | 80 | 51 | 1.6 | 111 | 12 | 6.2 | 3.5 | 60 | 66 | 7.0 | 83 | 105 | 147 | 4.1 |
| Day 1 | 0.2 | 8.0 | 95 | 45 | 1.6 | 131 | 9.0 | 6.4 | 3.9 | 63 | 74 | 3.8 | 89 | 113 | 151 | 4.4 |
| Day 15 | 0.1 | 7.0 | 86 | 87 | 1.8 | 78 | 16 | 6.7 | 3.4 | 63 | 90 | 6.4 | 154 | 111 | 151 | 5.1 |
| Day 43 | 0.1 | 8.0 | 89 | 87 | 1.5 | 77 | 13 | 6.1 | 3.6 | 67 | 65 | 5.8 | 203 | 111 | 148 | 4.0 |

Open Label Sheep
Midazolam Sheep #457, Tazz dose 15 mg/day
Hematology

| | | | | | | | | Platlet | | | | | | |
|---|---|---|---|---|---|---|---|---|---|---|---|---|---|---|
| | | | | | | | | Count | | | | | | |
| | | | | | | | | ×10³/ | Differential Count: | | | | | |
| | WBC | RBC | Hgb | Hct | MCV | MCH | MCHC | μl | q Absolute Values % | | | | | |
| | ×10³/μl | ×10⁶/μl | g/dl | % | fl | pg | g/dl | 250- | Mono | Lymph | Seg | Eosin | Atyp | |
| Day | 4-12 | 9-15 | 9-15 | 27-45 | 28-40 | 8-12 | 31-34 | 750 | 0-6 | 40-75 | 10-50 | 0-10 | lym | Bands |
| Baseline | 16.1 | 10.53 | 11.6 | 37.40 | 32.3 | 10 | 31.0 | adequate | 1 | 61 | 21 | 17 | | |
| Day 1 | 11.9 | 8.68 | 10.5 | 34.4 | 33.4 | 10.2 | 30.5 | 276 | | 26 | 73 | 1 | | |
| Day 15 | 15.4 | 8.72 | 10.9 | 35.4 | 34.4 | 10.6 | 30.8 | 45 | 3 | 71 | 24 | | | |
| Day 43 | 11.2 | 10.98 | 12.6 | 40.4 | 33.7 | 11.5 | 31.2 | Un- | 2 | 68 | 23 | 7 | | |
| | | | | | | | | available* | | | | | | |

*Platelet clumps observed

Chemistries

| | Total | | | Alk | Crea- | Glu- | | Total | Albu- | | Gamma | | | | | |
|---|---|---|---|---|---|---|---|---|---|---|---|---|---|---|---|---|
| | Bili | | | Phos | tinine | cose | BUN | Prot | min | Choles- | GT | Phos | | Cl− | Na+ | K+ |
| | mg/dl | ALT | AST | U/L | mg/dl | mg/dl | mg/dl | g/dl | g/dl | terol | U/L | mg/dl | CPK | mEq/l | mEq/l | mEq/l |
| Day | 0.14-0.32 | U/L | U/L | 68-387 | 1-2.7 | 42-76 | 8-20 | 6-7.9 | 2.4-3 | mg/dl | 25-59 | 5-7.3 | U/L | 95-103 | 139-152 | 3.9-5.4 |
| Day 1 | 0.1 | 10 | 85 | 43 | 1.1 | 91 | 14 | 5.0 | 2.8 | 59 | 59 | 6.2 | 122 | 108 | 147 | 4.4 |
| Day 15 | 0.1 | 14 | 55 | 93 | 1.1 | 60 | 20 | 5.9 | 3.3 | 65 | 72 | 4.7 | 73 | 111 | 150 | 4.4 |
| Day 43 | 0.1 | 12 | 69 | 105 | 1.4 | 67 | 15 | 5.7 | 3.3 | 88 | 61 | 5.9 | 51 | 107 | 145 | 4.9 |

Open Label Sheep
Midazolam Sheep #83, Wacko dose 15 mg/day
Hematology

| | | | | | | | | Platlet | | | | | | |
|---|---|---|---|---|---|---|---|---|---|---|---|---|---|---|
| | | | | | | | | Count | | | | | | |
| | | | | | | | | ×10³/ | Differential Count: | | | | | |
| | WBC | RBC | Hgb | Hct | MCV | MCH | MCHC | μl | q Absolute Values % | | | | | |
| | ×10³/μl | ×10⁶/μl | g/dl | % | fl | pg | g/dl | 250- | Mono | Lymph | Seg | Eosin | | Atyp |
| Day | 4-12 | 9-15 | 9-15 | 27-45 | 28-40 | 8-12 | 31-34 | 750 | 0-6 | 40-75 | 10-50 | 0-10 | Baso | lym |
| Baseline | 6.03 | 9.95 | 11.8 | 34.6 | 34.8 | 11.9 | 34.1 | 657 | 4 | 58 | 37 | | 1 | |
| Day 1 | 5.32 | 11.2 | 12.7 | 37.2 | 33.2 | 11.3 | 34.0 | 879 | 5 | 55 | 39 | 1 | | |
| Day 15 | 4.39 | 10.0 | 11.5 | 33.2 | 33.0 | 11.4 | 34.7 | 296 | 2 | 68 | 29 | 1 | | |
| Day 43 | 4.77 | 10.7 | 11.9 | 34.9 | 32.5 | 11.1 | 31.1 | 576 | 3 | 70 | 22 | 3 | | 2 |

Chemistries

| | Total | | | Alk | Crea- | Glu- | | Total | Albu- | | Gamma | | | | | |
|---|---|---|---|---|---|---|---|---|---|---|---|---|---|---|---|---|
| | Bili | | | Phos | tinine | cose | BUN | Prot | min | Choles- | GT | Phos | | Cl− | Na+ | K+ |
| | mg/dl | ALT | AST | U/L | mg/dl | mg/dl | mg/dl | g/dl | g/dl | terol | U/L | mg/dl | CPK | mEq/l | mEq/l | mEq/l |
| Day | 0.14-0.32 | U/L | U/L | 68-387 | 1-2.7 | 42-76 | 8-20 | 6-7.9 | 2.4-3 | mg/dl | 25-59 | 5-7.3 | U/L | 95-103 | 139-152 | 3.9-5.4 |
| Day 1 | 0.1 | 0 | 102 | 48 | 1.4 | 78 | 8 | 6.1 | 3.3 | 47 | 62 | 7.1 | 65 | 110 | 148 | 4.2 |
| Day 15 | 0.1 | 8 | 52 | 90 | 1.2 | 66 | 90 | 5.9 | 3.3 | 65 | 72 | 5.1 | 107 | 110 | 147 | 4.5 |
| Day 43 | 0.1 | 6 | 53 | 96 | 1.3 | 83 | 14 | 6.6 | 3.6 | 52 | 54 | 6.2 | 49 | 109 | 149 | 4.9 |

Open Label Sheep
Midazolam Sheep #91, Yacko dose 15 mg/day
Hematology

| | | | | | | | | Platlet | | | | | | |
|---|---|---|---|---|---|---|---|---|---|---|---|---|---|---|
| | | | | | | | | Count | | | | | | |
| | | | | | | | | ×10³/ | Differential Count: | | | | | |
| | WBC | RBC | Hgb | Hct | MCV | MCH | MCHC | μl | q Absolute Values % | | | | | |
| | ×10³/μl | ×10⁶/μl | g/dl | % | fl | pg | g/dl | 250- | Mono | Lymph | Seg | Eosin | | Atyp |
| Day | 4-12 | 9-15 | 9-15 | 27-45 | 28-40 | 8-12 | 31-34 | 750 | 0-6 | 40-75 | 10-50 | 0-10 | Baso | lym |
| Baseline | 11.2 | 9.51 | 10.1 | 31.3 | 32.9 | 10.6 | 32.3 | 817 | 5 | 22 | 73 | | 1 | |
| Day 1 | 7.09 | 9.18 | 9.84 | 29.2 | 31.8 | 10.7 | 33.7 | 996 | 2 | 21 | 76 | 1 | | |
| Day 15 | 5.13 | 10.0 | 10.8 | 31.8 | 31.8 | 10.8 | 34.0 | 848 | | 44 | 44 | 12 | | |
| Day 43 | 5.33 | 11.8 | 12.2 | 36.3 | 30.9 | 10.4 | 33.6 | 805 | 2 | 41 | 52 | 5 | | |

Chemistries

| | Total | | | Alk | Crea- | Glu- | | Total | Albu- | | Gamma | | | | | |
|---|---|---|---|---|---|---|---|---|---|---|---|---|---|---|---|---|
| | Bili | | | Phos | tinine | cose | BUN | Prot | min | Choles- | GT | Phos | | Cl− | Na+ | K+ |
| | mg/dl | ALT | AST | U/L | mg/dl | mg/dl | mg/dl | g/dl | g/dl | terol | U/L | mg/dl | CPK | mEq/l | mEq/l | mEq/l |
| Day | 0.14-0.32 | U/L | U/L | 68-387 | 1-2.7 | 42-76 | 8-20 | 6-7.9 | 2.4-3 | mg/dl | 25-59 | 5-7.3 | U/L | 95-103 | 139-152 | 3.9-5.4 |
| Day 1 | 0.1 | 7 | 61 | 132 | 1.6 | 77 | 9 | 6.1 | 3.3 | 47 | 51 | 6.2 | 52 | 112 | 149 | 4.9 |
| Day 15 | 0.0 | 10 | 50 | 162 | 1.3 | 83 | 13 | 6.2 | 3.3 | 56 | 60 | 6.1 | 56 | 108 | 148 | 4.4 |
| Day 43 | 0.1 | 9 | 63 | 174 | 1.5 | 81 | 14 | 6.4 | 3.6 | 66 | 58 | 7.3 | 54 | 106 | 147 | 4.6 |

Open Label Sheep
Midazolam Sheep #85, Dot dose 15 mg/day|
Hematology

| | | | | | | | | Platlet | | | | | | |
|---|---|---|---|---|---|---|---|---|---|---|---|---|---|---|
| | | | | | | | | Count | | | | | | |
| | | | | | | | | ×10³/ | Differential Count: | | | | | |
| | WBC | RBC | Hgb | Hct | MCV | MCH | MCHC | μl | q Absolute Values % | | | | | |
| | ×10³/μl | ×10⁶/μl | g/dl | % | fl | pg | g/dl | 250- | Mono | Lymph | Seg | Eosin | | Atyp |
| Day | 4-12 | 9-15 | 9-15 | 27-45 | 28-40 | 8-12 | 31-34 | 750 | 0-6 | 40-75 | 10-50 | 0-10 | Baso | lym |
| Baseline | 5.13 | 10.2 | 10.6 | 33.3 | 32.7 | 10.4 | 31.9 | 838 | | 61 | 37 | | 1 | 1 |
| Day 1 | 4.78 | 8.57 | 9.2 | 28.0 | 32.7 | 10.7 | 32.8 | 994 | 1 | 22 | 75 | 1 | 1 | |
| Day 15 | 5.13 | 10.0 | 10.8 | 31.8 | 31.8 | 10.8 | 34.0 | 848 | | 44 | 44 | 12 | | |
| Day 43 | 3.13 | 9.44 | 11.9 | 34.7 | 36.8 | 12.6 | 34.1 | 604 | 3 | 75 | 18 | 4 | | |

Chemistries

| | Total | | | Alk | Crea- | Glu- | | Total | Albu- | | Gamma | | | | | |
|---|---|---|---|---|---|---|---|---|---|---|---|---|---|---|---|---|
| | Bili | | | Phos | tinine | cose | BUN | Prot | min | Choles- | GT | Phos | | Cl− | Na+ | K+ |
| | mg/dl | ALT | AST | U/L | mg/dl | mg/dl | mg/dl | g/dl | g/dl | terol | U/L | mg/dl | CPK | mEq/l | mEq/l | mEq/l |
| Day | 0.14-0.32 | U/L | U/L | 68-387 | 1-2.7 | 42-76 | 8-20 | 6-7.9 | 2.4-3 | mg/dl | 25-59 | 5-7.3 | U/L | 95-103 | 139-152 | 3.9-5.4 |
| Day 1 | 0.1 | 7 | 76 | 50 | 1.4 | 75 | 10 | 6.1 | 3.4 | 52 | 68 | 5.3 | 99 | 114 | 149 | 4.7 |
| Day 15 | 0.0 | 10 | 56 | 54 | 1.2 | N/A | 17 | 5.4 | 2.8 | 58 | 54 | 6.0 | 56 | 101 | 148 | 5.0 |
| Day 43 | 0.1 | 8 | 59 | 92 | 1.3 | 76 | 13 | 6.4 | 3.6 | 72 | 60 | 7.1 | 81 | 108 | 148 | 4.9 |

Open Label Sheep
CSF Fluid Analysis and Routine Cultures

| | | | RBC Count | | WBC Count | | Total Protein | | Glucose | | Routine | |
|---|---|---|---|---|---|---|---|---|---|---|---|---|
| | Gross Exam | | (/μl) | | (/μl) | | (mg/dl) | | (mg/dl) | | Cultures | |
| | Peri- | Post | Peri- | Post | Peri- | Post | Peri- | Post | Peri- | Post | | Post |
| Sheep | operative | Mortum | operative | Mortum | operative | Mortum | operative | Mortum | operative | Mortum | Pre-op | Mortum |
| Red | color- | colorless, | 38 | 772 | 0 | 4 | N/A | N/A | N/A | N/A | No | Sample |
| #473 | less, | clear | | | | | | | | | Growth | con- |
| | clear | | | | | | | | | | | taminated |
| | | | | | | | | | | | | at |
| | | | | | | | | | | | | collection |
| Toad | N/A | colorless, | N/A | 2160 | N/A | 36 | N/A | 39.6 | N/A | 40 | No | No |
| #498 | | clear | | | | | | | | | Growth | Growth |
| Sandy | N/A | colorless, | N/A | 56 | N/A | 206 | N/A | 46.1 | N/A | 36 | No | No |
| #518 | | clear | | | | | | | | | Growth | Growth |
| Tazz | N/A | colorless, | N/A | 113 | N/A | 49 | N/A | 28 | N/A | 37 | No | No |
| #457 | | clear | | | | | | | | | Growth | Growth |
| Wacko | color- | colorless, | N/A | 134 | color- | 11 | 23 | 37 | N/A | 43 | No | No |
| #83 | less, | clear | | | less, | | | | | | Growth | Growth |
| | clear | | | | clear | | | | | | | |
| Yacko | color- | colorless, | 550 | 625 | 3 | 10 | 43 | 51.2 | 59 | 39 | No | No |
| #91 | less, | clear | | | | | | | | | Growth | Growth |
| | clear | | | | | | | | | | | |
| Dot | color- | colorless, | 250 | 20 | N/A | 4 | 24 | 29.8 | 44 | 30 | No | No |
| #85 | less, | clear | | | | | | | | | Growth | Growth |
| | clear | | | | | | | | | | | |

### EXAMPLE 2

### Toxicity and Nociceptive Testing of Midazolam in the Acute Pain Sheep Model—Closed Label trial

As in Example 1, a closed-label (investigators blinded as to dose) study was subsequently performed with 8 sheep which were administered 5 mg/day (N=3), 15 mg/day (N=3), or saline control (N=2). In this study, all sheep again exhibited significant pain relief. Five and 15 mg/day of intrathecal midazolam produced increases in pain tolerance from 10 to 100% of maximal possible effect in most animals. The continuous infusion of midazolam in the closed label sheep did not produce any behavioral, toxicological, or histopathologic changes related to the midazolam infusion in any of the animals studied.

Double Blinded Sheep
Sheep: Chicken Hawk Ovine #87
Midazolam dose: 5 mg/day

| | | | | | | | | Body |
|---|---|---|---|---|---|---|---|---|
| | | | | | | | Pulse | Temp. |
| | Mechanical | Allodynia | | Gait | Blood | | Rate | Normal |
| | Stimulus | Warm | Cold | Normal | pressure | | Normal | 101.3- |
| Day | % MPE | 50° C. | 6° C. | 0 | Sys | Dia | 55-115 | 104.0 |
| Baseline | — | 13 | 84 | 0 | 124 | 50 | 94 | 102.2 |
| Day 1 | 53.14 | 8 | 64 | 0 | 112 | 62 | 75 | 102.4 |
| Day 3 | −1.61 | 0 | 3 | 0 | 127 | 37 | 83 | 102.6 |
| Day 7 | −43.07 | 7 | 9 | 0 | 122 | 53 | 63 | 102.8 |
| Day 15 | 100 | 22 | 35 | 0 | 132 | 51 | 101 | 102.2 |
| Day 22 | 100 | 11 | 10 | 0 | 130 | 44 | 79 | 102.6 |
| Day 29 | 100 | 0 | 0 | 0 | 126 | 65 | 88 | 102.0 |
| Day 36 | 100 | 0 | 4 | 0 | 151 | 69 | 84 | 102.2 |
| Day 43 | 100 | 2 | 17 | 0 | 130 | 48 | 82 | 102.6 |

Necropsy: catheter placement confirmed as intrathecal, no gross lesions noted. Histology: mild catheter reaction.

Double Blinded Sheep
Sheep: Wiley Coyote Ovine #464
Midazolam dose: Control

| | | | | | | | | Body |
|---|---|---|---|---|---|---|---|---|
| | | | | | | | Pulse | Temp. |
| | Mechanical | Allodynia | | Gait | Blood | | Rate | Normal |
| | Stimulus | Warm | Cold | Normal | pressure | | Normal | 101.3- |
| Day | % MPE | 55° C. | 6° C. | 0 | Sys | Dia | 55-115 | 104.0 |
| Baseline | — | 109 | 48 | 0 | 133 | 74 | 117 | 102.0 |
| Day 1 | 72.70 | 4 | 7 | 0 | 157 | 76 | 72 | 104.6 |
| Day 3 | 65.98 | 7 | 4 | 0 | 152 | 50 | 83 | 101.2 |
| Day 7 | 93.97 | 8 | 11 | 0 | 150 | 57 | 67 | 102.4 |
| Day 15 | 4.39 | 123 | 32 | 0 | 132 | 56 | 84 | 102.4 |
| Day 22 | 33.68 | 2 | 1 | 0 | 131 | 58 | 83 | 102.0 |
| Day 29 | 27.30 | 17 | 8 | 0 | 120 | 38 | 70 | 102.0 |
| Day 36 | 100 | 2 | 0 | 0 | 136 | 49 | 86 | 102.8 |
| Day 43 | 1.2 | 79 | 7 | 0 | 115 | 45 | 91 | 101.4 |

*Battery failure in pump, pump replaced with new pump.
Necropsy: catheter placement confirmed as intrathecal, no gross lesions noted. Histology: mild catheter reaction.

Double Blinded Sheep
Sheep: Roadrunner Ovine #84
Midazolam dose: 15 mg/day

| | | | | | | | | Body |
|---|---|---|---|---|---|---|---|---|
| | | | | | | | Pulse | Temp. |
| | Mechanical | Allodynia | | Gait | Blood | | Rate | Normal |
| | Stimulus | Warm | Cold | Normal | pressure | | Normal | 101.3- |
| Day | % MPE | 55° C. | 6° C. | 0 | Sys | Dia | 55-115 | 104.0 |
| Baseline | — | 83 | 137 | 0 | 146 | 44 | 107 | 101.8 |
| Day 1 | −11.40 | 4 | 13 | 0 | 137 | 75 | 90 | 101.8 |
| Day 3 | 10.57 | 13 | 1 | 0 | 137 | 77 | 97 | 101.6 |
| Day 7 | −4.83 | 28 | 45 | 0 | 124 | 49 | 104 | 102.2 |
| Day 15 | −5.25 | 33 | 17 | 0 | 132 | 77 | 78 | 104.0 |
| Day 22 | 17.75 | 26 | 7 | 0 | 116 | 42 | 94 | 102.4 |
| Day 29 | 8.63 | 46 | 3 | 0 | 137 | 50 | 130 | 102.0 |
| Day 36 | 8.70 | 8 | 4 | 0 | 137 | 51 | 124 | 101.8 |
| Day 43 | 2.35 | 7 | 1 | 0 | 144 | 40 | 77 | 102.6 |

Necropsy: catheter placement confirmed as intrathecal, no gross lesions noted. Histology: mild catheter reaction.

Double Blinded Sheep
Sheep: Porky Ovine #402
Midazolam dose: 5 mg/day

| | | | | | | | | Body |
|---|---|---|---|---|---|---|---|---|
| | | | | | | | Pulse | Temp. |
| | Mechanical | Allodynia | | Gait | Blood | | Rate | Normal |
| | Stimulus | Warm | Cold | Normal | pressure | | Normal | 101.3- |
| Day | % MPE | 55° C. | 6° C. | 0 | Sys | Dia | 55-115 | 104.0 |
| Baseline | — | 57 | 89 | 0 | 151 | 76 | 102 | 101.4 |
| Day 1 | 61.99 | 10 | 55 | 0 | 148 | 65 | 87 | 102.0 |
| Day 3 | 71.35 | 62 | 41 | 0 | 107 | 70 | 99 | 102.6 |
| Day 7 | 17.94 | 32 | 28 | 0 | 140 | 42 | 89 | 102.0 |
| Day 15 | 100 | 3 | 23 | 0 | 133 | 67 | 100 | 101.0 |
| Day 22 | 77.60 | 82 | 27 | 0 | 113 | 55 | 90 | 102.6 |
| Day 29 | 86.71 | 46 | 24 | 0 | 135 | 68 | 86 | 101.2 |
| Day 36 | −3.70 | 32 | 43 | 0 | 134 | 41 | 87 | 102.0 |
| Day 43 | 77.17 | 8 | 12 | 0 | 147 | 40 | 108 | 102.2 |

Necropsy: catheter placement confirmed as intrathecal, no gross lesions noted. Histology: mild catheter reaction.

Double Blinded Sheep
Sheep: Petunia Ovine #439
Midazolam dose: 15 mg/day

| | | | | | | | | Body |
|---|---|---|---|---|---|---|---|---|
| | | | | | | | Pulse | Temp. |
| | Mechanical | Allodynia | | Gait | Blood | | Rate | Normal |
| | Stimulus | Warm | Cold | Normal | pressure | | Normal | 101.3- |
| Day | % MPE | 55° C. | 6° C. | 0 | Sys | Dia | 55-115 | 104.0 |
| Baseline | — | 2 | 1 | 0 | 138 | 26 | 108 | 102.0 |
| Day 1 | 0.46 | 30 | 9 | 0 | 133 | 34 | 89 | 102.0 |
| Day 3 | −35.29 | 41 | 2 | 0 | 152 | 49 | 72 | 102.8 |
| Day 7 | 1.24 | 0 | 0 | 0 | 126 | 44 | 74 | 102.0 |
| Day 15 | 31.27 | 0 | 0 | 0 | 132 | 32 | 69 | 102.0 |
| Day 22 | −102.63 | 3 | 14 | 0 | 123 | 26 | 66 | 103.0 |
| Day 29 | 50.31 | 38 | 1 | 0 | 114 | 32 | 63 | 101.0 |
| Day 36 | −161.15 | 67 | 0 | 0 | 120 | 45 | 71 | 102.0 |
| Day 43 | 48.45 | 0 | 0 | 0 | 142 | 49 | 90 | 102.4 |

Necropsy: catheter placement confirmed as intrathecal, no gross lesions noted. Histology: mild catheter reaction.

Double Blinded Sheep
Sheep: Elmer Fudd Ovine #437
Midazolam dose: 5 mg/day

| | | | | | | | | Body |
|---|---|---|---|---|---|---|---|---|
| | | | | | | | Pulse | Temp. |
| | Mechanical | Allodynia | | Gait | Blood | | Rate | Normal |
| | Stimulus | Warm | Cold | Normal | pressure | | Normal | 101.3- |
| Day | % MPE | 55° C. | 6° C. | 0 | Sys | Dia | 55-115 | 104.0 |
| Baseline | — | 26 | 6 | 0 | 161 | 60 | 76 | 102.0 |
| Day 1 | 54.51 | 2 | 5 | 0 | 122 | 68 | 87 | 102.2 |
| Day 3 | 82.22 | 2 | 2 | 0 | 138 | 64 | 70 | 102.2 |
| Day 7 | 52.39 | 7 | 6 | 0 | 119 | 51 | 80 | 102.8 |
| Day 15 | 10.50 | 0 | 0 | 0 | 131 | 40 | 70 | 101.4 |
| Day 22 | 83.41 | 2 | 2 | 0 | 150 | 70 | 83 | 102.2 |
| Day 29 | 67.50 | 3 | 0 | 0 | 136 | 43 | 79 | 102.0 |
| Day 36 | 100 | 0 | 0 | 0 | 139 | 31 | 73 | 101.8 |
| Day 43 | 4.35 | 4 | 4 | 0 | 120 | 56 | 85 | 102.0 |

Necropsy: catheter placement confirmed as intrathecal, no gross lesions noted. Histology: mild catheter reaction.

Double Blinded Sheep
Sheep: Marvin Martian Ovine #814
Midazolam dose: Control

| | | | | | | | | Body |
|---|---|---|---|---|---|---|---|---|
| | | | | | | | Pulse | Temp. |
| | Mechanical | Allodynia | | Gait | Blood | | Rate | Normal |
| | Stimulus | Warm | Cold | Normal | pressure | | Normal | 101.3- |
| Day | % MPE | 55° C. | 6° C. | 0 | Sys | Dia | 55-115 | 104.0 |
| Baseline | — | 103 | 32 | 0 | 139 | 64 | 99 | 103.0 |
| Day 1 | 9.86 | 80 | 5 | 0 | 110 | 59 | 60 | 103.4 |
| Day 3 | 13.25 | 40 | 6 | 0 | 130 | 66 | 96 | 103.2 |
| Day 7 | −40.74 | 1 | 5 | 0 | 129 | 57 | 86 | 103.0 |
| Day 15 | −19.49 | 3 | 0 | 0 | 160 | 69 | 98 | 102.4 |
| Day 22 | −13.06 | 39 | 0 | 0 | 130 | 58 | 103 | 103.0 |
| Day 29 | 39.07 | 45 | 4 | 0 | 144 | 76 | 91 | 101.8 |
| Day 36 | −43.71 | 12 | 2 | 0 | 146 | 62 | 96 | 102.8 |
| Day 43 | 100 | 1 | 2 | 0 | 151 | 65 | 87 | 102.8 |

Necropsy: catheter placement confirmed as intrathecal, no gross lesions noted. Histology: no histologic changes were observed.

Double Blinded Sheep
Sheep: Leghorn II Ovine #815
Midazolam dose: 15 mg/day

| | | | | | | | | Body |
|---|---|---|---|---|---|---|---|---|
| | | | | | | | Pulse | Temp. |
| | Mechanical | Allodynia | | Gait | Blood | | Rate | Normal |
| | Stimulus | Warm | Cold | Normal | pressure | | Normal | 101.3- |
| Day | % MPE | 50° C. | 6° C. | 0 | Sys | Dia | 55-115 | 104.0 |
| Baseline | — | 107 | 171 | 0 | 95 | 63 | 114 | 102.4 |
| Day 1 | 33.49 | 78 | 16* | 0 | 130 | 67 | 111 | 104.2 |
| Day 3 | 3.95 | 65 | 3 | 0 | 152 | 58 | 144 | 102.4 |
| Day 7 | 100 | 5 | 12 | 0 | 143 | 70 | 88 | 102.4 |
| Day 15 | 24.84 | 14 | 0 | 0 | 152 | 71 | 125 | 102.8 |
| Day 22 | 14.02 | 2 | 26 | 0 | 160 | 87 | 121 | 102.4 |
| Day 29 | 66.23 | 0 | 0 | 0 | 138 | 43 | 97 | 102.8 |
| Day 36 | 100 | 2 | 2 | 0 | 145 | 66 | 89 | 102.2 |
| Day 43 | 47.41 | 22 | 29 | 0 | 130 | 35 | 87 | 102.6 |

Necropsy: catheter placement confirmed as intrathecal. No obvious lesions where detected in the spinal cord, meninges or vertebral canal associated with the intrathecal catheter. Histology: mild catheter reaction.

Double Blinded Sheep
Sheep: Chicken Hawk, ovine #87
Dose: 5 mg/day
Hematology

| | | | | | | | | Platlet | | | | | | |
|---|---|---|---|---|---|---|---|---|---|---|---|---|---|---|
| | | | | | | | | Count | | | | | | |
| | | | | | | | | ×10³/ | Differential Count: | | | | | |
| | WBC | RBC | Hgb | Hct | MCV | MCH | MCHC | μl | q Absolute Values % | | | | | |
| | ×10³/μl | ×10⁶/μl | g/dl | % | fl | Pg | g/dl | 250- | Mono | Lymph | Seg | | Eosin | Atyp |
| Day | 4-12 | 9-15 | 9-15 | 27-45 | 28-40 | 8-12 | 31-34 | 750 | 0-6 | 40-75 | 10-50 | Baso | 0-10 | lym |
| Baseline | 6.18 | 9.77 | 10.8 | 31.5 | 32.3 | 11.0 | 34.2 | 848 | 4 | 41 | 53 | 0 | 2 | 0 |
| Day 1 | 6.00 | 11.0 | 11.7 | 34.1 | 30.19 | 30.9 | 34.3 | 1280 | 7 | 55 | 36 | 0 | 2 | 0 |
| Day 15 | 5.69 | 9.70 | 10.0 | 29.8 | 30.8 | 10.4 | 33.6 | 1018 | 4 | 62 | 29 | 1 | 4 | 0 |
| Day 43 | 5.13 | 10.2 | 10.7 | 32.3 | 31.7 | 10.5 | 33.0 | 891 | 3 | 53 | 37 | 1 | 5 | 1 |

Chemistries

| | Total | | | Alk | Crea- | Glu- | | Total | Albu- | | Gamma | | | | | |
|---|---|---|---|---|---|---|---|---|---|---|---|---|---|---|---|---|
| | Bili | | | Phos | tinine | cose | BUN | Prot | min | Choles- | GT | Phos | | Cl− | Na+ | K+ |
| | mg/dl | ALT | AST | U/L | mg/dl | mg/dl | mg/dl | g/dl | g/dl | terol | U/L | mg/dl | CPK | mEq/l | mEq/l | mEq/l |
| Day | 0.14-0.32 | U/L | U/L | 68-387 | 1-2.7 | 42-76 | 8-20 | 6-7.9 | 2.4-3 | mg/dl | 25-59 | 5-7.3 | U/L | 95-103 | 139-152 | 3.9-5.4 |
| Day 1 | 0.1 | 4 | 63 | 54 | 1.3 | 67 | 14 | 5.7 | 3.2 | 71 | 55 | 6.6 | 163 | 112 | 150 | 4.5 |
| Day 15 | 0.1 | 6 | 58 | 79 | 1.3 | 79 | 17 | 6.2 | 3.8 | 61 | 64 | 5.0 | 425 | 109 | 148 | 4.7 |
| Day 43 | 0.1 | 8 | 62 | 149 | 1.1 | 82 | 14 | 6.5 | 3.8 | 62 | 55 | 6.5 | 61 | 108 | 147 | 5.0 |

Double Blinded Sheep
Sheep: Wiley Coyote Ovine #464
Control
Hematology

| | | | | | | | | Platlet | | | | | | | |
|---|---|---|---|---|---|---|---|---|---|---|---|---|---|---|---|
| | | | | | | | | Count | | | | | | | |
| | | | | | | | | ×10³/ | Differential Count: | | | | | | |
| | WBC | RBC | Hgb | Hct | MCV | MCH | MCHC | μl | q Absolute Values % | | | | | | |
| | ×10³/μl | ×10⁶/μl | g/dl | % | fl | Pg | G/dl | 250- | | Mono | Lymph | Seg | Eosin | Atyp | Baso |
| Day | 4-12 | 9-15 | 9-15 | 27-45 | 28-40 | 8-12 | 31-34 | 750 | Bands | 0-6 | 40-75 | 10-50 | 0-10 | lym | 0-3 |
| Baseline | 6.32 | 11.8 | 13.4 | 38.4 | 32.2 | 11.3 | 35.1 | 990 | 0 | 4 | 33 | 39 | 24 | 0 | 0 |
| Day 1 | 5.58 | 10.9 | 12.3 | 35.8 | 32.9 | 11.3 | 34.3 | 836 | 0 | 4 | 36 | 57 | 3 | 0 | 0 |
| Day 15 | 5.21 | 12.0 | 13.0 | 38.1 | 31.8 | 10.8 | 34.1 | 919 | 0 | 5 | 41 | 40 | 13 | 0 | |
| Day 43 | 4.13 | 12.4 | 13.2 | 38.9 | 31.5 | 10.7 | 33.9 | 776 | 0 | 3 | 56 | 36 | 4 | 0 | |

Chemistries

| | Total | | | Alk | Crea- | Glu- | BUN | Total | Albu- | | Gamma | Phos | | | | |
|---|---|---|---|---|---|---|---|---|---|---|---|---|---|---|---|---|
| | Bili | | | Phos | tinine | Cose | Mg/ | Prot | min | Choles- | GT | Mg/ | | Cl− | Na+ | K+ |
| | mg/dl | ALT | AST | U/L | mg/dl | mg/dl | dl | g/dl | g/dl | terol | U/L | dl | CPK | mEq/l | mEq/l | mEq/l |
| Day | 0.14-0.32 | U/L | U/L | 68-387 | 1-2.7 | 42-76 | 8-20 | 6-7.9 | 2.4-3 | Mg/dl | 25-59 | 5-7.3 | U/L | 95-103 | 139-152 | 3.9-5.4 |
| Day 1 | 0.1 | 18 | 125 | 74 | 1.3 | 103 | 10 | 6.1 | 3.2 | 51 | 44 | 5.6 | 196 | 113 | 151 | 45 |
| Day 15 | 0.1 | 3 | 59 | 115 | 1.4 | 84 | 16 | 6.2 | 3.4 | 41 | 51 | 7.4 | 135 | 110 | 150 | 4.7 |
| Day 43 | 0.1 | 9 | 68 | 151 | 1.1 | 75 | 16 | 6.0 | 3.4 | 72 | 47 | 6.0 | 61 | 111 | 147 | 5.5 |

Double Blinded Sheep
Midazolam Sheep #84, Roadrunner dose 15 mg/day-
Hematology

| | | | | | | | | Platlet | | | | | | |
|---|---|---|---|---|---|---|---|---|---|---|---|---|---|---|
| | | | | | | | | Count | | | | | | |
| | | | | | | | | ×10³/ | Differential Count: | | | | | |
| | WBC | RBC | Hgb | Hct | MCV | MCH | MCHC | μl | q Absolute Values % | | | | | |
| | ×10³/μl | ×10⁶/μl | g/dl | % | fl | pg | g/dl | 250- | Mono | Lymph | Seg | Eosin | | Atyp |
| Day | 4-12 | 9-15 | 9-15 | 27-45 | 28-40 | 8-12 | 31-34 | 750 | 0-6 | 40-75 | 10-50 | 0-10 | Baso | lym |
| Baseline | 5.79 | 14.0 | 15.0 | 41.9 | 30.0 | 10.7 | 35.7 | 721 | 1 | 62 | 32 | 5 | | |
| Day 1 | 5.39 | 11.7 | 12.9 | 35.7 | 30.4 | 11.0 | 36.1 | 962 | 3 | 43 | 46 | 8 | | |
| Day 15 | 5.13 | 10.0 | 10.8 | 31.8 | 31.8 | 10.8 | 34.0 | 848 | | 44 | 44 | 12 | | |
| Day 43 | 5.15 | 10.1 | 11.9 | 35.0 | 34.7 | 11.8 | 34.0 | 727 | 1 | 67 | 29 | 2 | | 1 |

Chemistries

| | Total | | | Alk | Crea- | Glu- | | Total | Albu- | | Gamma | | | | | |
|---|---|---|---|---|---|---|---|---|---|---|---|---|---|---|---|---|
| | Bili | | | Phos | tinine | cose | BUN | Prot | min | Choles- | GT | Phos | | Cl− | Na+ | K+ |
| | mg/dl | ALT | AST | U/L | mg/dl | mg/dl | mg/dl | g/dl | g/dl | terol | U/L | mg/dl | CPK | mEq/l | mEq/l | mEq/l |
| Day | 0.14-0.32 | U/L | U/L | 68-387 | 1-2.7 | 42-76 | 8-20 | 6-7.9 | 2.4-3 | mg/dl | 25-59 | 5-7.3 | U/L | 95-103 | 139-152 | 3.9-5.4 |
| Day 1 | 0.3 | 9 | 100 | 93 | 1.2 | 69 | 7 | 5.9 | 3.3 | 45 | 51 | 6.2 | 90 | 111 | 148 | 4.8 |
| Day 15 | 0.1 | 2 | 60 | 98 | 1.3 | 66 | 11 | 6.4 | 3.7 | 56 | 62 | 6.6 | 53 | 111 | 150 | 4.8 |
| Day 43 | 0.1 | 7 | 65 | 143 | 1.2 | 74 | 11 | 6.0 | 3.54 | 57 | 56 | 7.1 | 102 | 108 | 149 | 4.6 |

Double Blinded Sheep
Sheep: Porky Ovine #402
Dose: 5 mg/day
Hematology

| | | | | | | | | Platlet | | | | | | |
|---|---|---|---|---|---|---|---|---|---|---|---|---|---|---|
| | | | | | | | | Count | | | | | | |
| | | | | | | | | ×10³/ | Differential Count: | | | | | |
| | WBC | RBC | Hgb | Hct | MCV | MCH | MCHC | μl | q Absolute Values % | | | | | |
| | ×10³/μl | ×10⁶/μl | g/dl | % | fl | Pg | g/dl | 250- | Mono | Lymph | Seg | Eosin | Atyp | |
| Day | 4-12 | 9-15 | 9-15 | 27-45 | 28-40 | 8-12 | 31-34 | 750 | 0-6 | 40-75 | 10-50 | 0-10 | lym | Bands |
| Baseline | 3.53 | 10.9 | 13.5 | 40.7 | 37.2 | 12.4 | 33.2 | 215 | 2 | 48 | 48 | 2 | 0 | 0 |
| Day 1 | 3.47 | 10.9 | 13.4 | 40.4 | 37.1 | 12.3 | 33.1 | 319 | 5 | 41 | 53 | 1 | 0 | 0 |
| Day 15 | 3.17 | 8.23 | 10.2 | 31.4 | 38.2 | 12.3 | 32.3 | 173 | 5 | 33 | 61 | 1 | 0 | 0 |
| Day 43 | 3.59 | 10.4 | 12.2 | 39.2 | 37.6 | 12.0 | 31.8 | 214 | 5 | 40 | 49 | 5 | 1 | 0 |

Chemistries

| | Total | | | Alk | Crea- | Glu- | | Total | Albu- | | Gamma | | | | | |
|---|---|---|---|---|---|---|---|---|---|---|---|---|---|---|---|---|
| | Bili | | | Phos | tinine | cose | BUN | Prot | min | Choles- | GT | Phos | | Cl− | Na+ | K+ |
| | mg/dl | ALT | AST | U/L | mg/dl | mg/dl | mg/dl | g/dl | g/dl | terol | U/L | mg/dl | CPK | mEq/l | mEq/l | mEq/l |
| Day | 0.14-0.32 | U/L | U/L | 68-387 | 1-2.7 | 42-76 | 8-20 | 6-7.9 | 2.4-3 | mg/dl | 25-59 | 5-7.3 | U/L | 95-103 | 139-152 | 3.9-5.4 |
| Day 1 | 0 | 3 | 67 | 37 | 1.3 | 72 | 10 | 6.8 | 3.5 | 71 | 46 | 7.5 | 73 | 115 | 153 | 4.6 |
| Day 15 | 0.2 | 8 | 69 | 33 | 1.2 | 89 | 14 | 7.4 | 3.9 | 3.5 | 74 | 48 | 94 | 112 | 150 | 4.2 |
| Day 43 | 0.1 | 13 | 59 | 55 | 1.3 | 61 | 17 | 6.4 | 3.5 | 62 | 42 | 5.9 | 93 | 108 | 147 | 4.8 |

Double Blinded Sheep
Sheep: Petunia Ovine #439
Dose 15 mg/day
Hematology

| | | | | | | | | Platlet | | | | | | |
|---|---|---|---|---|---|---|---|---|---|---|---|---|---|---|
| | | | | | | | | Count | | | | | | |
| | | | | | | | | ×10³/ | Differential Count: | | | | | |
| | WBC | RBC | Hgb | Hct | MCV | MCH | MCHC | μl | q Absolute Values % | | | | | |
| | ×10³/μl | ×10⁶/μl | g/dl | % | fl | pg | G/dl | 250- | Mono | Lymph | Seg | Eosin | Atyp | |
| Day | 4-12 | 9-15 | 9-15 | 27-45 | 28-40 | 8-12 | 31-34 | 750 | 0-6 | 40-75 | 10-50 | 0-10 | lym | Bands |
| Baseline | 3.12 | 12.2 | 14.0 | 41.1 | 33.6 | 11.5 | 34.2 | 489 | 7 | 53 | 27 | 130 | 0 | 0 |
| Day 1 | 2.92 | 12.7 | 14.4 | 42.4 | 33.3 | 11.3 | 33.9 | 715• | 6 | 50 | 40 | 1 | 2 | 1 |
| Day 15 | 3.6 | 9.02 | 10.1 | 30.8 | 34.2 | 11.2 | 32.7 | 606• | 3 | 46 | 48 | 2 | 1 | 0 |
| Day 43 | 2.3 | 10.5 | 11.8 | 36.0 | 34.1 | 11.2 | 32.7 | 356 | 4 | 47 | 41 | 7 | 1 | 0 |

•Platelet clumps observed

Chemistries

| | Total | | | Alk | Crea- | Glu- | | Total | Albu- | | Gamma | | | | | |
|---|---|---|---|---|---|---|---|---|---|---|---|---|---|---|---|---|
| | Bili | | | Phos | tinine | cose | BUN | Prot | min | Choles- | GT | Phos | | Cl− | Na+ | K+ |
| | mg/dl | ALT | AST | U/L | mg/dl | mg/dl | mg/dl | g/dl | g/dl | terol | U/L | mg/dl | CPK | mEq/l | mEq/l | mEq/l |
| Day | 0.14-0.32 | U/L | U/L | 68-387 | 1-2.7 | 42-76 | 8-20 | 6-7.9 | 2.4-3 | mg/dl | 25-59 | 5-7.3 | U/L | 95-103 | 139-152 | 3.9-5.4 |
| Day 1 | 0 | 0 | 102 | 43 | 1.1 | 71 | 10 | 6.6 | 3.6 | 60 | 84 | 7.9 | 84 | 112 | 152 | 5.4 |
| Day 15 | 0.3 | 1 | 94 | 29 | 1.2 | 93 | 14 | 7.2 | 4.0 | 61 | 74 | 4.9 | 105 | 110 | 150 | 4.3 |
| Day 43 | 0.1 | 0 | 97 | 49 | 1.2 | 68 | 17 | 6.3 | 3.6 | 65 | 77 | 6.2 | 91 | 110 | 146 | 5.2 |

Double Blinded Sheep
Sheep: Elmer Fudd Ovine #437
Dose 5 mg/day
Hematology

| | | | | | | | | Platlet | | | | | | |
|---|---|---|---|---|---|---|---|---|---|---|---|---|---|---|
| | | | | | | | | Count | | | | | | |
| | | | | | | | | ×10³/ | Differential Count: | | | | | |
| | WBC | RBC | Hgb | Hct | MCV | MCH | MCHC | μl | q Absolute Values % | | | | | |
| | ×10³/μl | ×10⁶/μl | g/dl | % | fl | pg | G/dl | 250- | Mono | Lymph | Seg | Eosin | Atyp | |
| Day | 4-12 | 9-15 | 9-15 | 27-45 | 28-40 | 8-12 | 31-34 | 750 | 0-6 | 40-75 | 10-50 | 0-10 | lym | Bands |
| Baseline | 5.32 | 11.2 | 13.4 | 38.5 | 34.5 | 12.0 | 34.7 | 562 | 9 | 35 | 51 | 54 | 0 | 0 |
| Day 1 | 6.6 | 10.8 | 12.4 | 36.1 | 33.4 | 11.4 | 34.3 | 788 | 6 | 31 | 61 | 1 | 0 | 1 |
| Day 15 | 4.99 | 11.4 | 12.7 | 37.3 | 32.8 | 11.2 | 34.1 | 677 | 2 | 39 | 49 | 10 | 0 | 0 |
| Day 43 | 4.65 | 10.9 | 11.8 | 34.6 | 31.7 | 10.8 | 34.2 | 540 | 1 | 38 | 59 | 2 | 0 | 0 |

Chemistries

| | Total | | | Alk | Crea- | Glu- | | Total | Albu- | | Gamma | | | | | |
|---|---|---|---|---|---|---|---|---|---|---|---|---|---|---|---|---|
| | Bili | | | Phos | tinine | cose | BUN | Prot | min | Choles- | GT | Phos | | Cl− | Na+ | K+ |
| | mg/dl | ALT | AST | U/L | mg/dl | mg/dl | mg/dl | g/dl | g/dl | terol | U/L | mg/dl | CPK | mEq/l | mEq/l | mEq/l |
| Day | 0.14-0.32 | U/L | U/L | 68-387 | 1-2.7 | 42-76 | 8-20 | 6-7.9 | 2.4-3 | mg/dl | 25-59 | 5-7.3 | U/L | 95-103 | 139-152 | 3.9-5.4 |
| Day 1 | 0.1 | 8 | 70 | 49 | 1.3 | 104 | 17 | 7.4 | 4.1 | 78 | 51 | 5.9 | 49 | 109 | 150 | 4.7 |
| Day 15 | 0.1 | 6 | 67 | 88 | 1.2 | 73 | 15 | 6.8 | 3.8 | 74 | 54 | 6.6 | 77 | 109 | 149 | 4.7 |
| Day 43 | 0.1 | 9 | 61 | 72 | 1.3 | 83 | 16 | 6.6 | 4.1 | 90 | 48 | 6.2 | 64 | 108 | 147 | 4.3 |

Double Blinded Sheep
Sheep: Marvin Martian Ovine #814
Control
Hematology

| | | | | | | | | Platlet | | | | | | |
|---|---|---|---|---|---|---|---|---|---|---|---|---|---|---|
| | | | | | | | | Count | | | | | | |
| | | | | | | | | ×10³/ | Differential Count: | | | | | |
| | WBC | RBC | Hgb | Hct | MCV | MCH | MCHC | μl | q Absolute Values % | | | | | |
| | ×10³/μl | ×10⁶/μl | g/dl | % | fl | pg | G/dl | 250- | Mono | Lymph | Seg | Eosin | Atyp | |
| Day | 4-12 | 9-15 | 9-15 | 27-45 | 28-40 | 8-12 | 31-34 | 750 | 0-6 | 40-75 | 10-50 | 0-10 | lym | Baso |
| Baseline | 6.95 | 10.6 | 11.6 | 33.9 | 31.8 | 10.9 | 34.2 | 512 | 1 | 67 | 19 | 8 | 0 | 1 |
| Day 1 | 7.95 | 11.1 | 11.8 | 34.0 | 30.6 | 10.6 | 34.6 | 853• | 3 | 58 | 37 | 1 | 0 | 1 |
| Day 15 | 5.10 | 9.49 | 10.3 | 30.0 | 31.7 | 10.9 | 34.4 | 691 | 3 | 64 | 24 | 8 | 0 | 1 |
| Day 43 | 6.9 | 9.63 | 10.4 | 30.5 | 31.7 | 10.8 | 33.9 | 664 | 0 | 67 | 28 | 5 | 0 | 0 |

•Platelet clumps observed

Chemistries

| | Total | | | Alk | Crea- | Glu- | | Total | Albu- | | Gamma | | | | | |
|---|---|---|---|---|---|---|---|---|---|---|---|---|---|---|---|---|
| | Bili | | | Phos | tinine | cose | BUN | Prot | min | Choles- | GT | Phos | | Cl− | Na+ | K+ |
| | mg/dl | ALT | AST | U/L | mg/dl | mg/dl | mg/dl | g/dl | g/dl | terol | U/L | mg/dl | CPK | mEq/l | mEq/l | mEq/l |
| Day | 0.14-0.32 | U/L | U/L | 68-387 | 1-2.7 | 42-76 | 8-20 | 6-7.9 | 2.4-3 | mg/dl | 25-59 | 5-7.3 | U/L | 95-103 | 139-152 | 3.9-5.4 |
| Day 1 | 0.1 | 6 | 81 | 82 | 1.0 | 100 | 10 | 6.9 | 3.8 | 74 | 45 | 6.0 | 55 | 111 | 149 | 4.3 |
| Day 15 | 0.1 | 7 | 76 | 198 | 0.9 | 85 | 9 | 5.9 | 3.4 | 79 | 46 | 8.7 | 77 | 108 | 146 | 4.8 |
| Day 43 | 0.1 | 9 | 83 | 179 | 1.0 | 94 | 12 | 5.9 | 4.0 | 78 | 50 | 9.7 | 69 | | 147 | 4.9 |

Double Blinded Sheep
Sheep: Leghorn II. Ovine #815
Dose 15 mg/day
Hematology

| | | | | | | | | Platlet | | | | | | |
|---|---|---|---|---|---|---|---|---|---|---|---|---|---|---|
| | | | | | | | | Count | | | | | | |
| | | | | | | | | ×10³/ | Differential Count: | | | | | |
| | WBC | RBC | Hgb | Hct | MCV | MCH | MCHC | μl | q Absolute Values % | | | | | |
| | ×10³/μl | ×10⁶/μl | g/dl | % | fl | pg | G/dl | 250- | Mono | Lymph | Seg | Eosin | Atyp | |
| Day | 4-12 | 9-15 | 9-15 | 27-45 | 28-40 | 8-12 | 31-34 | 750 | 0-6 | 40-75 | 10-50 | 0-10 | lym | Baso |
| Baseline | 6.73 | 11.7 | 12.5 | 36.9 | 31.5 | 10.7 | 33.9 | 516 | 3 | 79 | 14 | 4 | 0 | 0 |
| Day 1 | 6.07 | 11.1 | 11.8 | 34.7 | 31.1 | 10.6 | 34.0 | 828 | 2 | 58 | 28 | 2 | 0 | 0 |
| Day 15 | 6.64 | 12.0 | 12.4 | 38.0 | 31.8 | 10.4 | 32.7 | 861 | 1 | 59 | 24 | 16 | 0 | 0 |
| Day 43 | 6.11 | 11.7 | 12.3 | 36.4 | 31.2 | 10.5 | 33.7 | 345 | 2 | 77 | 18 | 2 | 0 | 1 |

Chemistries

| | Total | | | Alk | Crea- | Glu- | | Total | Albu- | | Gamma | | | | | |
|---|---|---|---|---|---|---|---|---|---|---|---|---|---|---|---|---|
| | Bili | | | Phos | tinine | cose | BUN | Prot | min | Choles- | GT | Phos | | Cl− | Na+ | K+ |
| | mg/dl | ALT | AST | U/L | mg/dl | mg/dl | mg/dl | g/dl | g/dl | terol | U/L | mg/dl | CPK | mEq/l | mEq/l | mEq/l |
| Day | 0.14-0.32 | U/L | U/L | 68-387 | 1-2.7 | 42-76 | 8-20 | 6-7.9 | 2.4-3 | mg/dl | 25-59 | 5-7.3 | U/L | 95-103 | 139-152 | 3.9-5.4 |
| Day 1 | 0 | 14 | 117 | 117 | 0.9 | 80 | 19 | 6.3 | 3.5 | 68 | 80 | 8.3 | 94 | 108 | 146 | 5.0 |
| Day 15 | 0.1 | 10 | 72 | 154 | 1.1 | 82 | 25 | 7.1 | 3.6 | 74 | 87 | 6.4 | 100 | 106 | 146 | 4.8 |
| Day 43 | 0.1 | 15 | 78 | 179 | 1.0 | 70 | 26 | 6.9 | 3.6 | 80 | 73 | 6.9 | 96 | 109 | 146 | 5.7 |

CSF Fluid Analysis and Routine Cultures
Double Blinded Study

| | | | RBC Count | | WBC Count | | Total Protein | | Glucose (mg/dl) | | Routine | |
|---|---|---|---|---|---|---|---|---|---|---|---|---|
| | Gross Exam | | (/μl) | | (/μl) | | (mg/dl) | | | Post | Cultures | |
| | Peri- | Post | Peri- | Post | Peri- | Post | Peri- | Post | Peri- | Mor- | | Post |
| Sheep | operative | Mortum | operative | Mortum | operative | Mortum | operative | Mortum | operative | tum | Pre-op | Mortum |
| Chicken | Colorless | Colorless | 3 | 4 | 1 | 4 | 31 | 39 | 38 | 47 | No | No |
| Hawk #87 | clear | clear | | | | | | | | | Growth | Growth |
| Wiley | Colorless | Colorless | 1735* | 1325 | 1 | 29 | 47 | 42 | 50 | 34 | No | No |
| Coyote #464 | clear | cloudy | | | | | | | | | Growth | Growth |
| Roadrunner | Colorless | Colorless | 2615* | 480 | 1 | 12 | 45 | 73 | 47 | 35 | No | No |
| #84 | clear | cloudy | | | | | | | | | Growth | Growth |
| Porky | Colorless | Colorless | 785 | 725 | 1 | 80 | 42.7 | 40 | 51 | 25 | No | No |
| #402 | clear | clear | | | | | | | | | Growth | Growth |
| Petunia | Colorless | Pale pink | 10 | 4250 | 1 | 22 | 45.1 | 47 | 73 | 31 | No | No |
| #439 | clear | Cloudy** | | | | | | | | | Growth | Growth |
| Elmer Fudd | Colorless | *** | 1 | | 0 | | 46 | | 68 | | | |
| #437 | clear | | | | | | | | | | | |
| Marvin | Colorless | Colorless | 14 | 63 | 0 | 8 | 24 | 35 | 68 | 38 | No | No |
| Martian | clear | clear | | | | | | | | | Growth | Growth |
| #814 | | | | | | | | | | | | |
| Leghorn II | Colorless | Colorless | 9 | 2 | 1 | 1 | 50 | 59 | 56 | 24 | No | No |
| #815 | clear | clear | | | | | | | | | Growth | Growth |
| (repeat of | | | | | | | | | | | | |
| Leghorn) | | | | | | | | | | | | |
| #88 | | | | | | | | | | | | |

*Nicked a vessel when threading catheter cephalad into the subarachnoid space (blood in CSF)
**Sample contaminated with blood during collection
***Not able to obtain sample

### EXAMPLE 3

### Toxicity Testing of Midazolam in the Pig Model—Closed Label Trial

Another closed label study was performed in pigs. This study was performed in response to implications of possible species-related toxicity reported by researchers in Turkey and France (Malinovsky et al., 1991; Svensson et al., 1995; Erdine et al, 1999). These three studies demonstrated neurotoxicity in the rat and rabbit models following intrathecal bolus doses of midazolam. Rats were administered single or multiple bolus doses for 20 days. Rabbits were administered bolus injections of 0.1% midazolam (pH 3.3) in 0.3 ml or preservative-free midazolam (pH 3.5) in 0.3 ml for one day or five consecutive days. To further assess the safety of spinally administered midazolam by continuous infusion in a second species, three pigs were instrumented with Medtronic SynchroMed® intrathecal infusion systems. Pigs were administered 15 mg/day (N=2), or saline control (N=1). No clinical or gross changes at necropsy were observed in any animal. Histology revealed a foreign body reaction to the catheter, however, there was no evidence of toxicity related to the midazolam infusion.

MIDAZOLAM SWINE DATA

| | Pre-drug | Final | Clinical | | |
|---|---|---|---|---|---|
| Name | Weight | Weight | Symptoms | Drug Dose | Histopathological Changes |
| Percy | 104 | 147 | none | 15 mg | Most foreign body reaction in all the pigs. |
| Pete | 121 | 170 | none | 15 mg | The least foreign body reaction in all the pigs. The spinal reaction |
| | | | | | was more severe than any of the sheep seen in this protocol; but less |
| | | | | | severe than the other pigs. |
| Prudence | 104 | 161 | none | Control | Second most foreign body reaction in all the pigs. |

| | | | RBC Count | | WBC Count | | Total Protein | | Glucose (mg/dl) | | | |
|---|---|---|---|---|---|---|---|---|---|---|---|---|
| | Gross Exam | | (/ul) | | (/ul) | | (mg/dl) | | | Post | Routine Cultures | |
| | Peri- | Post | Peri- | Post | Peri- | Post | Peri- | Post | Peri- | Mor- | | Post |
| Swine | operative | Mortum | operative | Mortum | operative | Mortum | operative | Mortum | operative | tum | Pre-op | Mortum |
| Percy | Colorless | Colorless | 49 | 1,350 | 1 | 888 | 40 | 73 | 63 | 11 | No | No |
| | clear | cloudy | | | | | | | | | Growth | Growth |
| Pete | Pink, | Colorless | 10,950 | 0 | 11 | 117 | 53 | 39 | 51 | 31 | No | No |
| | cloudy | clear | | | | | | | | | Growth | Growth |
| Prudence | Pink, | Colorless | 2,990 | 7 | 51 | 316 | 59 | 54 | Not | 30 | No | No |
| | cloudy | clear | | | | | | | run | | Growth | Growth |

### EXAMPLE 4

### Testing of the Neuropathic Pain Model in Sheep

The neuropathic pain model further defined the efficacy of a new preservative-free formulation for the treatment of neuropathic pain before proceeding to clinical trials in subjects with this syndrome. The efficacy and toxicity of continuous infusion intrathecal midazolam in the sheep model of neuropathic pain was determined.

The neuropathic pain model in the sheep is created by placing four tight ligatures around the median, radial, or ulnar nerve (or a combination thereof) with 0-chromic gut suture. This method produces a chronic painful peripheral mononeuropathy which may be related to those conditions seen in humans with causalgia and reflex sympathetic dystrophy. To date, the onset of neuropathic pain has occurred 1 to 9 days postoperatively, as evidenced by the display of painful behavior such as hyperalgesia, not bearing weight or holding the operated leg off the ground. The duration of the painful behavior lasted 16 to 62 days. However, three of the eight animals studied thus far did not develop neuropathic pain after observation for up to 43 days. It has been difficult to identify a segment of the median nerve that is consistently appropriate to achieve the neuropathic pain behavior. The inventors are continuing to test different segments of the median nerve and other mixed sensory/motor nerves to consistently produce this behavior. Of the five animals that developed neuropathic pain, three were treated with intrathecal morphine or midazolam as single agents.

In the sheep with neuropathic pain treated by midazolam alone, intrathecal administration of midazolam was initiated at 3 to 5 mg/day and the dose was escalated to up to 15 mg/day. Midazolam alone produced significant analgesia. This analgesia was documented using a mechanical stimulus device and/or behavior monitoring. Results of monitoring by the mechanical stimulus device are shown in the following table for Sheep #819. The mechanical stimulus device measures pressure applied to a blunt pin against the foreleg. The pressure at which the animal lifts its leg in response to the painful stimulus is documented. In the sheep monitored by this device, administration of intrathecal midazolam allowed endurance of up to 18 Newtons of pressure (cut off to prevent tissue damage was set at 19.99 N) compared to the baseline (pretreatment) pressure endured of 9.59 N.

### EXAMPLE 5

### Evaluation of Behavior in the Neuropathic Pain Sheep Model

To evaluate animal behavior, the Observer 3.0 software (Noldus Information Technology, The Netherlands) was used to aid docmentation of a daily record of multiple traits. Percentages calculated reflect the percentage of time that the animal displayed a certain behavior during a 15-minute observation period per day, administered at the same time each day. The behavior was then interpreted into a visual analog pain score (VAS), where 0 represents no pain and 100 represents severe pain. Results are shown in the following tables. On most days, VAS scores averaged approximately 30 while receiving intrathecal midazolam 3 to 6 mg/day compared to pretreatment scores during saline treatment from 60 to 95 (midazolam-naive).

In addition to the increase in pain tolerance documented by the mechanical stimulus device, the VAS score for the first sheep (Rowdy) averaged 30% while receiving midazolam 5 mg/day. Higher doses appeared to produce less analgesia in this animal as evidenced by signs of increased pain after dose escalation to 15 mg/day. Midazolam was discontinued in this animal and treatment was initiated with intrathecal morphine/clonidine. Following 6 days of treatment with morphine the sheep began to limp on the right rear leg and began biting herself and pulling wool out of her skin. This behavior continued sporadically the remaining 13 days of treatment with morphine/clonidine. Gross and microscopic evaluation of the spinal tissue revealed swelling and inflammation surrounding the catheter tip which was located on the right lateral side and produced mild to moderate spinal cord compression. Development of inflammatory lesions is consistent with our previous animal studies investigating the toxicity of intrathecal morphine.

In a second sheep (# 980 Dudley), treatment of neuropathic pain was initially begun with morphine 1 mg/day, which was gradually increased to 6 mg/day without adequate pain relief (average pain score during this period of 76%). After 13 days of morphine treatment, the dose was increased to 6.5 mg/day which provided sufficient relief (pain score of 20%). The morphine was stopped to re-establish the neuropathic pain syndrome, which returned in 9 days. At this time, 3 mg/day of midazolam alone was initiated for 13 days and provided significant pain relief (average pain score of 32%). To again re-establish the neuropathic pain syndrome, the midazolam infusion was stopped. Neuropathic pain returned in 3 days, and midazolam 3 mg/day reinstituted. Midazolam dose was escalated to 5 mg/day over a 7 day period without adequate pain relief (average pain score 65%). On the eighth day, the dose was increased to 6 mg/day which produced adequate pain relief for the remainder of the study (average pain score 35%). At necropsy, this animal did not exhibit any spinal cord lesions associated with the administration of midazolam. It is also important to note that no significant inflammatory lesions were observed in any sheep receiving midazolam alone in the acute pain sheep study.

A third animal (# 604 Clint) exhibiting neuropathic pain received morphine alone. For the first 13 days of morphine infusion at 6 mg/day, the pain scored average 43%. The morphine was then stopped on several occasions with return of neuropathic pain. Infusions begun with either morphine or saline resulted in average pain scores between 30-50%. However, while on morphine therapy the animal exhibited restlessness, pain behaviors such as biting itself near the area of the catheter tip, and frequently laying down. These behaviors would subside somewhat during saline infusion periods. At necropsy, a cavitated lesion in the spinal cord in front of the catheter tip was found, measuring 0.8 cm (L)×0.5 cm (W)×0.3 cm (Depth).

Data from these animals demonstrate that intrathecal midazolam up to 5 mg/day had analgesic activity against neuropathic pain and was tolerated well as an alternative to morphine. It is possible that higher doses of midazolam could produce some degree of hyperalgesia, thereby reducing analgesic effects. Further study may help refine optimal dosages.

Sheep-Rowdy-#819

| | | | | | | | Holding | | | | | |
|---|---|---|---|---|---|---|---|---|---|---|---|---|
| | | | | Holding | | | un- | | | | | |
| | | | | operated | | | operated | | Mechan- | | | Res- |
| | | | | leg | | | leg | | ical | Blood | | pirations |
| | | Pain | | off the | Walk- | Stand- | off the | Other | Stimulus | pre- | | per |
| Date | Dose | Score | Eating | ground | ing | ing | ground | behavior | device | assure | Pulse | minute |
| Baseline | | | | | | | | | 9.59N | 120/69 | 71 | 24 |
| May 19, 2001 | saline | 65 | 41.53% | 6.75% | 4.32% | 43.96% | 3.43% | | | | | 40 |
| May 20, 2001 | saline | 75 | 0.09% | 20.11% | 0.98% | 76.23% | 2.60% | | | | | * |
| May 21, 2001 | saline | 60 | 76.56% | 2.04% | 2.09% | 10.31% | 6.46% | Laying | | | | 44 |
| | | | | | | | | down | | | | |
| | | | | | | | | 1.73% | | | | |
| May 22, 2001 | saline | 95 | 47.69% | 8.05% | 2.02% | 20.74% | 4.51% | Not | 7.08N | 145/74 | 91 | 24 |
| | | | | | | | | bearing | | | | |
| | | | | | | | | wieght | | | | |
| | | | | | | | | on | | | | |
| | | | | | | | | operated | | | | |
| | | | | | | | | leg | | | | |
| | | | | | | | | 16.98% | | | | |
| May 23, 2001 | 5 mg | 30 | 80.63% | 1.50% | 5.84% | 10.13% | 1.90% | | 18.0N | 144/82 | 104 | 20 |
| May 24, 2001 | 5 mg | 10 | 84.36% | 2.49% | 4.56% | 7.79% | 0.24% | Observe | | | | 44 |
| | | | | | | | | operated | | | | |
| | | | | | | | | leg | | | | |
| | | | | | | | | 0.56% | | | | |
| May 25, 2001 | 5 mg | 30 | 65.42% | 3.16% | 6.39% | 22.77% | 2.26% | | 13.5N | 158/90 | 93 | 40 |
| May 26, 2001 | 5 mg | 25 | 45.60% | 3.60% | | 50.42% | 0.39% | | | | | 48 |
| May 28, 2001 | 5 mg | 35 | 81.67% | 2.86% | 0.92% | 13.19% | 1.35% | | | | | 60 |
| May 29, 2001 | 5 mg | 50 | 69.88% | 5.77% | 5.07% | 18.38% | 0.90% | | 11.45N | 135/75 | 106 | 40 |
| May 30, 2001 | 5 mg | 20 | 83.94% | 6.24% | 4.22% | 5.05% | 0.56% | | | | | 56 |
| May 31, 2001 | saline | 80 | 45.42% | 11.48% | 7.06% | 21.65% | 2.68% | Laying | 4.86N | 129/68 | 112 | 52 |
| | | | | | | | | down | | | | |
| | | | | | | | | 1.23%, | | | | |
| | | | | | | | | wagging | | | | |
| | | | | | | | | tail | | | | |
| | | | | | | | | 0.44% | | | | |
| Jun. 1, 2001 | 10 | 50 | 74.01% | 6.06% | 6.05% | 12.71% | 0.88% | Eating | 13.7N | 136/75 | 122 | 36 |
| | mg | | | | | | | lying | | | | |
| | | | | | | | | down | | | | |
| | | | | | | | | 0.29% | | | | |
| Jun. 2, 2001 | 10 | 70 | | | | | | | | | | 80 |
| | mg | | | | | | | | | | | |
| Jun. 3, 2001 | 10 | 70 | | | | | | | | | | 60 |
| | mg | | | | | | | | | | | |
| Jun. 4, 2001 | 10 | 60 | 85.68% | 6.30% | 6.86% | 0.38% | 0.79% | | | | | 36 |
| | mg | | | | | | | | | | | |
| Jun. 5, 2001 | 10 | 60 | 43.56% | 7.45% | 16.10% | 31.75% | 1.14% | | | | | |
| | mg | | | | | | | | | | | |
| Jun. 6, 2001 | saline | 97 | 0 | 36.97% | 2.06% | 31.53% | 2.68% | Standing | 7.8N | 142/78 | 94 | 44 |
| | | | | | | | | ruminat- | | | | |
| | | | | | | | | ing | | | | |
| | | | | | | | | 27.17% | | | | |
| Jun. 7, 2001 | 15 | 40 | 3.29% | 8.55% | 1.40% | 13.79% | 0.81% | Standing | 7.0N | 143/77 | 94 | 40 |
| | mg | | | | | | | ruminat- | | | | |
| | | | | | | | | ing | | | | |
| | | | | | | | | 70.19% | | | | |
| | | | | | | | | Pulling | | | | |
| | | | | | | | | wool | | | | |
| | | | | | | | | 1.11% | | | | |
| | | | | | | | | Bleating | | | | |
| | | | | | | | | 0.86% | | | | |

*observation failure
N = Newtons

Sheep #980: Dudley

| | | | | | | | | Holding | Not | | | | | |
|---|---|---|---|---|---|---|---|---|---|---|---|---|---|---|
| | | | | | Holding | | | un- | bearing | | | | | |
| | | | | | operated | | | operated | weight | | | | | |
| | Dose | | | | leg | | | leg | on | Observe | | | Other | Resp |
| | per | Study | Pain | | off the | Walk- | Stand- | off the | operated | Operated | Laying | Grind | be- | per |
| Date | day | Day | Score | Eating | ground | ing | ing | ground | leg | leg | down | teeth | havior | min. |
| Base- | sa- | 0 | 90% | 0 | 2.51% | 4.43% | 0.10% | 2.51% | 76.3% | 0 | 2.89% | 12.59% | 0 | 20 |
| line | line | | | | | | | | | | | | | |
| Aug. 23,2002 | MS | 1 | 90% | 1.47% | 26.59% | 2.79% | 0 | 0.105% | 64.84% | 0.96% | 0 | 3.24% | | 22 |
| | 1 mg | | | | | | | | | | | | | |
| Aug. 24, 2002 | MS | 2 | 83% | 1.55% | 17.75% | 0.735% | 20.72% | 0.22% | 55.68% | 0 | 0 | 2.85% | Drink | 43 |
| | 1 mg | | | | | | | | | | | | 0.5% | |
| Aug. 25, 2002 | MS | 3 | 78% | 7.36% | 3.12% | 1.17% | 3.15% | 0 | 85.03% | 0 | 0 | 0.19% | 0 | 40 |
| | 1 mg | | | | | | | | | | | | | |
| Aug. 26, 2002 | MS | 4 | 90% | 0 | 15.88% | 0.47% | 17.34% | 0 | 64.74% | 0.19% | 0 | 1.39% | 0 | 22 |
| | 2 mg | | | | | | | | | | | | | |
| Aug. 27, 2002 | MS | 5 | 85% | 7.76% | 21.62% | 3.64% | 0 | 0 | 60.52% | 0.47% | 0 | 3.3% | Rumi- | 20 |
| | 3 mg | | | | | | | | | | | | nating | |
| | | | | | | | | | | | | | standing | |
| | | | | | | | | | | | | | 2.69% | |
| Aug. 29, 2002 | MS | 7 | 85% | 0 | 20.91% | 1.16% | 0 | 0 | 77.18% | 0 | 0 | 0.76% | 0 | 24 |
| | 3 mg | | | | | | | | | | | | | |
| Aug. 30, 2002 | MS | 8 | 83% | 4.72% | 5.2% | 5.36% | 0 | 0 | 69.53% | 0 | 0 | 0 | Rumi- | 64 |
| | 4 mg | | | | | | | | | | | | nating | |
| | | | | | | | | | | | | | standing | |
| | | | | | | | | | | | | | 15.2% | |
| Aug. 31, 2002 | MS | 9 | 83% | 8.72% | 19.58% | 1.61% | 0 | 0 | 69.1% | 0.28% | 0 | 0 | Eating | |
| | 5 mg | | | | | | | | | | | | lying | |
| | | | | | | | | | | | | | 0.7%, | |
| | | | | | | | | | | | | | Rumi- | |
| | | | | | | | | | | | | | nating | |
| | | | | | | | | | | | | | standing | |
| | | | | | | | | | | | | | 0.1% | |
| Sept. 1, 2002 | MS | 10 | 85% | 0 | 3.52% | 2.05% | 0 | 0.19% | 85.9% | 0 | 1.59% | 0 | Rumi- | 28 |
| | 5 mg | | | | | | | | | | | | nating | |
| | | | | | | | | | | | | | standing | |
| | | | | | | | | | | | | | 5.65%, | |
| | | | | | | | | | | | | | pulling | |
| | | | | | | | | | | | | | wool | |
| | | | | | | | | | | | | | 0.28%, | |
| | | | | | | | | | | | | | pawing | |
| | | | | | | | | | | | | | ground | |
| | | | | | | | | | | | | | 0.85% | |
| Sept. 2, 2002 | MS | 11 | 85% | 0 | 13.34% | 3.83% | 0 | 0 | 72.14% | 0 | 0 | 0 | Rumi- | 32 |
| | 5 mg | | | | | | | | | | | | nating | |
| | | | | | | | | | | | | | standing | |
| | | | | | | | | | | | | | 9.7%, | |
| | | | | | | | | | | | | | bleating | |
| | | | | | | | | | | | | | 0.98% | |
| Sept. 3, 2002 | MS | 12 | 80% | 0 | 9.77% | 9.07% | 0.99% | 0.52% | 35.43% | 0.66% | 43.57% | 0 | 0 | 64 |
| | 6 mg | | | | | | | | | | | | | |
| Sept. 5, 2002 | MS | 14 | 90% | 0 | 0 | 0 | 0 | 0 | 0 | 0 | 100% | 0 | 0 | 24 |
| | 6.5 | | | | | | | | | | | | | |
| | mg | | | | | | | | | | | | | |
| Sept. 6, 2002 | MS | 15 | 73% | 15.7% | 3.53% | 3.72% | 10.8% | 0 | 66.17% | 0 | 0 | 0.24% | 0 | 31 |
| | 6.5 | | | | | | | | | | | | | |
| | mg | | | | | | | | | | | | | |
| Sept. 8, 2002 | MS | 17 | 20% | 35.29% | 0.18% | 0 | 0 | 0 | 51.43% | 0.47% | 0.6% | 0 | Eating | 28 |
| | 6.5 | | | | | | | | | | | | lying | |
| | mg | | | | | | | | | | | | 9.31% | |

MS: Morphine Sulfate

| | | | | | | | | Holding | Not | | | | | |
|---|---|---|---|---|---|---|---|---|---|---|---|---|---|---|
| | | | | | Holding | | | un- | bearing | | | | | |
| | | | | | operated | | | operated | weight | | | | | |
| | Dose | | | | leg | | | leg | on | Observe | | | Other | Resp |
| | per | Study | Pain | | off the | Walk- | Stand- | off the | operated | Operated | Laying | Grind | be- | per |
| Date | day | Day | Score | Eating | ground | ing | ing | ground | leg | leg | down | teeth | havior | min. |
| Sept. 9, 2002 | Stop- | 18 | 80% | 0 | | | 0 | | | 0 | 100% | 0 | Rumi- | 40 |
| | ped | | | | | | | | | | | | na- | |
| | pump | | | | | | | | | | | | ting | |
| | | | | | | | | | | | | | standing | |
| | | | | | | | | | | | | | 0.94% | |
| Sept. 10, 2002 | 0 | 19 | 15% | 20.42% | 1.87% | 13.56% | 0 | 0.67% | 62.54% | 0 | 0 | 0 | Rumi- | 32 |
| | | | | | | | | | | | | | nating | |
| | | | | | | | | | | | | | standing | |
| | | | | | | | | | | | | | 9.7%, | |
| | | | | | | | | | | | | | Bleating | |
| | | | | | | | | | | | | | 0.98% | |
| Sept. 11, 2002 | 0 | 20 | 65% | 0.44% | 11.36% | 0.73% | 36.77% | 0.17% | 49.27% | 0 | 0 | 1.26% | 0 | 35 |
| Sept. 12, 2002 | 0 | 21 | 25% | 47.16% | 1.93% | 0 | 0 | 0 | 50.74% | 0.17% | 0 | 0 | 0 | 26 |
| Sept. 13, 2002 | 0 | 22 | 40% | 31.35% | 4.08% | 1.25% | 0 | 0 | 52.26% | 1.01% | 0 | 0 | Eating | 24 |
| | | | | | | | | | | | | | lying | |
| | | | | | | | | | | | | | 10.06% | |
| Sept. 14, 2002 | 0 | 23 | 33% | 7.21% | 11.03% | 0.8% | 12.38% | 0 | 68.44% | 0 | 0 | 0.05% | Bleating | 36 |
| | | | | | | | | | | | | | 0.11% | |
| Sept. 15, 2002 | 0 | 24 | 35% | 0.77% | 0.38% | 1.12% | 0 | 0 | 97.73% | 0 | 0 | 0 | 0 | 36 |
| Sept. 16, 2002 | 0 | 25 | 40% | 15.72% | 5.81% | 2.64% | 0.18% | 0 | 65.76% | 1.3% | 8.39% | 0 | Rumi- | 35 |
| | | | | | | | | | | | | | nating | |
| | | | | | | | | | | | | | laying | |
| | | | | | | | | | | | | | down | |
| | | | | | | | | | | | | | 0.21% | |
| Sept. 17, 2002 | 0 | 26 | 70% | 0.28% | 14.88% | 4.36% | 0 | 0.67% | 65.37% | 9.12% | 0 | 5.33% | 0 | 24 |
| Sept. 18, 2002 | MDZ | 0 | 68% | 25.15% | 8.23% | 0.96% | 0 | 1.08% | 50.46% | 1.7% | 1.24% | 0 | Eating | 34 |
| | 2 mg | | | | | | | | | | | | lying | |
| | | | | | | | | | | | | | 1.69% | |
| Sept. 19, 2002 | MDZ | 1 | 73% | 23.81% | 3.1% | 2.85% | 0 | 0.39% | 39.78% | 1.05% | 31.54% | 0 | Drink- | 26 |
| | 2 mg | | | | | | | | | | | | ing | |
| | | | | | | | | | | | | | 0.6% | |
| Sept. 20, 2002 | MDZ | 2 | 30% | 37.18% | 0.21% | 2.63% | 30.48% | 0 | 19.93% | 0 | 8.76% | 0 | Drink- | 29 |
| | 2 mg | | | | | | | | | | | | ing | |
| | | | | | | | | | | | | | 0.17% | |
| | | | | | | | | | | | | | Defae- | |
| | | | | | | | | | | | | | cation | |
| | | | | | | | | | | | | | 0.65% | |
| Sept. 21, 2002 | MDZ | 3 | 48% | 43.1% | 37.84% | 0.65% | 0 | 0 | 16.08% | 0 | 2.35% | 0 | 0 | 30 |
| | 2 mg | | | | | | | | | | | | | |
| Sept. 22, 2002 | MDZ | 4 | 25% | 0 | 0 | 0 | 0 | 0 | 0 | 0 | 1.24% | 0 | Rumin- | 42 |
| | 2 mg | | | | | | | | | | | | ating | |
| | | | | | | | | | | | | | laying | |
| | | | | | | | | | | | | | down | |
| | | | | | | | | | | | | | 98.28%, | |
| | | | | | | | | | | | | | Rumi- | |
| | | | | | | | | | | | | | nating | |
| | | | | | | | | | | | | | standing | |
| | | | | | | | | | | | | | 0.48% | |
| Sept. 23, 2002 | MDZ | 5 | 20% | 0 | 0 | 100% | 0 | 0 | 0 | 0 | 0 | 0 | 0 | 28 |
| | 2 mg | | | | | | | | | | | | | |
| Sept. 24, 2002 | MDZ | 6 | 60% | 39.13% | 0 | 2.0% | 0 | 0 | 7.73% | 0 | 0 | 0 | Rumi- | 35 |
| | 3 mg | | | | | | | | | | | | nating | |
| | | | | | | | | | | | | | laying | |
| | | | | | | | | | | | | | down | |
| | | | | | | | | | | | | | 50%, | |
| | | | | | | | | | | | | | Eating | |
| | | | | | | | | | | | | | lying | |
| | | | | | | | | | | | | | 1.14% | |
| Sept. 25, 2002 | MDZ | 7 | 15% | 77.62% | 0 | 0.32% | 0 | 0 | 21.84% | 0.23% | 0 | 0 | 0 | 48 |
| | 3 mg | | | | | | | | | | | | | |
| Sept. 26, 2002 | MDZ | 8 | 23% | 34.30% | 0.72% | 0 | 33.05% | 0 | 29.65% | 0.12% | 2.18% | 0 | 0 | 60 |
| | 3 mg | | | | | | | | | | | | | |
| Sept. 27, 2002 | MDZ | 9 | 30% | 59.11% | 0.28% | 0.29% | 0 | 0 | 38.68% | 0.57% | 0 | 0 | Drink- | 50 |
| | 3 mg | | | | | | | | | | | | ing | |
| | | | | | | | | | | | | | 0.6% | |
| | | | | | | | | | | | | | Defae- | |
| | | | | | | | | | | | | | cation | |
| | | | | | | | | | | | | | 0.49% | |
| Sept. 30, 2002 | MDZ | 12 | 15% | 88.18% | 0.55% | 0.25% | 0 | 0 | 10.91% | 0 | 0.035% | 0 | 0 | 38 |
| | 3 mg | | | | | | | | | | | | | |
| Oct. 1, 2002 | Stop- | 0 | 15% | 79.26% | 0.77% | 2.31% | 1.33% | 0 | 15.8% | 0 | 0 | 0 | Defae- | 44 |
| | ped | | | | | | | | | | | | cation | |
| | pump | | | | | | | | | | | | 0.545% | |
| Oct. 2, 2002 | 0 | 1 | 35% | 20.23% | 1.95% | 1.5% | 1.34% | 0 | 49.84% | 0.15% | 0 | 0 | 0 | 64 |
| Oct. 4, 2002 | MDZ | 0 | 70% | 55.46% | 3.31% | 0.61% | 0 | 0 | 40.36% | 0.27% | 0 | 0 | 0 | 40 |
| | 3 mg | | | | | | | | | | | | | |
| Oct. 5, 2002 | MDZ | 1 | 60% | 0 | 3.54% | 0.08% | 0.31% | 0 | 96.07% | 0 | 0 | 0 | 0 | 72 |
| | 3 mg | | | | | | | | | | | | | |
| Oct. 6, 2002 | MDZ | 2 | 60% | 0 | 0 | 0 | 0 | 0 | 0 | 0 | 82.49% | 0 | Rumi- | 48 |
| | 3 mg | | | | | | | | | | | | nating | |
| | | | | | | | | | | | | | laying | |
| | | | | | | | | | | | | | down | |
| | | | | | | | | | | | | | 17.51% | |
| Oct. 7, 2002 | MDZ | 3 | 60% | 58.58% | 3.62% | 1.14% | 1.02% | 0 | 35.43% | 0 | 0 | 0 | 0 | 51 |
| | 3 mg | | | | | | | | | | | | | |
| Oct. 10, 2002 | MDZ | 6 | 50% | 36.49% | 2.78% | 1.09% | 0.52% | 0.36% | 29.65% | 1.51% | 0 | 0 | Bleating | 58 |
| | 4 mg | | | | | | | | | | | | 0.76% | |
| Oct. 12, 2002 | MDZ | 8 | 85% | 8.76% | 28.77% | 2.08% | 0 | 0 | 59.99% | 0 | 0 | 0 | Rumi- | 32 |
| | 5 mg | | | | | | | | | | | | nating | |
| | | | | | | | | | | | | | standing | |
| | | | | | | | | | | | | | 0.41% | |
| Oct. 13, 2002 | MDZ | 9 | 85% | 1.56% | 2.03% | 4.21% | 0 | 0 | 90.8% | 0 | 0 | 0 | Rumi- | 36 |
| | 5 mg | | | | | | | | | | | | nating | |
| | | | | | | | | | | | | | standing | |
| | | | | | | | | | | | | | 1.4% | |
| Oct. 14, 2002 | MDZ | 10 | 75% | 61.09% | 3.13% | 0.89% | 0 | 0 | 33.78% | 1.11% | 0 | 0 | 0 | 40 |
| | 6 mg | | | | | | | | | | | | | |
| Oct. 15, 2002 | MDZ | 11 | 28% | 73.59% | 0.71% | 0 | 0 | 0 | 22.96% | 0.36% | 0 | 0 | Pawing | 36 |
| | 6 mg | | | | | | | | | | | | ground | |
| | | | | | | | | | | | | | 0.78% | |
| | | | | | | | | | | | | | Eating | |
| | | | | | | | | | | | | | lying | |
| | | | | | | | | | | | | | 0.21% | |
| Oct. 16, 2002 | MDZ | 12 | 13% | 80.21% | 0.58% | 1.79% | 0 | 0 | 13.73% | 0.07% | 0 | 0 | Pawing | 42 |
| | 6 mg | | | | | | | | | | | | ground | |
| | | | | | | | | | | | | | 0.64%, | |
| | | | | | | | | | | | | | Pulling | |
| | | | | | | | | | | | | | wool | |
| | | | | | | | | | | | | | 0.26%, | |
| | | | | | | | | | | | | | Defae- | |
| | | | | | | | | | | | | | cation | |
| | | | | | | | | | | | | | 0.94% | |

Sheep #604 Clint

| | Day of | | |
|---|---|---|---|
| Date | Study | Dose per Day | Pain Score |
| Dec. 7, 2001 | 0 | 6 mg Morphine | 60% |
| Dec. 8, 2001 | 1 | 6 mg Morphine | 60% |
| Dec. 9, 2001 | 2 | 6 mg Morphine | 60% |
| Dec. 11, 2001 | 4 | 6 mg Morphine | 45% |
| Dec. 12, 2001 | 5 | 6 mg Morphine | 30% |
| Dec. 13, 2001 | 6 | 6 mg Morphine | 35% |
| Dec. 14, 2001 | 7 | 6 mg Morphine | 40% |
| Dec. 15, 2001 | 8 | 6 mg Morphine | 50% |
| Dec. 16, 2001 | 9 | 6 mg Morphine | 40% |
| Dec. 17, 2001 | 10 | 6 mg Morphine | 33% |
| Dec. 18, 2001 | 11 | 6 mg Morphine | 35% |
| Dec. 19, 2001 | 12 | 6 mg Morphine | 35% |
| Dec. 20, 2001 | 0 | 6 mg Morphine | 33% |
| | | | Changed to saline |
| | | | at 1:30 PM |
| Dec. 21, 2001 | 1 | Saline | 33% |
| Dec. 22, 2001 | 2 | Saline | 33% |
| Dec. 23, 2001 | 3 | Saline | 45% |
| Dec. 24, 2001 | 4 | Saline | 45% |
| Dec. 25, 2001 | 5 | Saline | 45% |
| Dec. 26, 2001 | 6 | Saline | 53% |
| Dec. 27, 2001 | 7 | Saline | 50% |
| Dec. 28, 2001 | 8 | Saline | 60% |
| | | | Changed to 6 mg |
| | | | Morphine at 2:48 PM |
| Dec. 29, 2001 | 1 | 6 mg Morphine | 50% |
| Dec. 30, 2001 | 2 | 6 mg Morphine | 40% |
| Dec. 31, 2001 | 3 | 6 mg Morphine | 38% |
| Jan. 1, 2002 | 4 | 6 mg Morphine | 40% |
| Jan. 2, 2002 | 5 | 6 mg Morphine | 30% |
| Jan. 3, 2002 | 6 | 6 mg Morphine | 33% |
| Jan. 4, 2002 | 7 | 6 mg Morphine | 30% |
| Jan. 5, 2002 | 8 | 6 mg Morphine | 30% |
| Jan. 6, 2002 | 9 | 6 mg Morphine | 30% |
| Jan. 7, 2002 | 10 | 6 mg Morphine | 30% |
| Jan. 8, 2002 | 11 | 6 mg Morphine | 30% |
| Jan. 9, 2002 | 12 | 6 mg Morphine | 15% |
| | | | Refilled pump with |
| | | | saline at 9:44 AM |
| Jan. 10, 2002 | 1 | Saline | 30% |
| Jan. 11, 2002 | 2 | Saline | 35% |
| Jan. 12, 2002 | 3 | Saline | 40% |
| Jan. 13, 2002 | 4 | Saline | 38% |
| Jan. 14, 2002 | 5 | Saline | 28% |
| Jan. 15, 2002 | 6 | Saline | 30% |
| Jan. 16, 2002 | 7 | Saline | 48% |
| Jan. 17, 2002 | 8 | Saline | 48% |
| Jan. 18, 2002 | 9 | Saline | 45% |
| Jan. 19, 2002 | 10 | Saline | 45% |
| Jan. 20, 2002 | 11 | Saline | 45% |
| Jan. 21, 2002 | 12 | Saline | 45% |
| Jan. 22, 2002 | 13 | Saline | 53% |
| Jan. 23, 2002 | 14 | Saline | 20% |
| Jan. 24, 2002 | 15 | Saline | 43% |
| Jan. 25, 2002 | 16 | Saline | 23% |
| Jan. 26, 2002 | 17 | Saline | 55% |
| Jan. 27, 2002 | 18 | Saline | 50% |
| Jan. 29, 2002 | 20 | Saline | 10% |
| Jan. 30, 2002 | 21 | Saline | 33% |
| Jan. 31, 2002 | 22 | Saline | 30% |
| Feb. 1, 2002 | 23 | Saline | 18% |
| Feb. 2, 2002 | 24 | Saline | 20% |
| Feb. 3, 2002 | 25 | Saline | 10% |
| Feb. 4, 2002 | 26 | Saline | 5% |
| Feb. 5, 2002 | 27 | Saline | 0% |
| | | | Neuropathic pain no |
| | | | longer present |

### EXAMPLE 6

### Experimental Procedures—Acute Pain Model

Testing of Midazolam Hydrochloride

Drug formulation and stability testing for preservative-free midazolam for intrathecal use was performed using HPLC with the final drug concentration at 2.5 or 5.00 mg/ml in normal saline (ingredients: NaCl 0.9% and 0.45% respectively). The concentrations of 2.5 and 5.0 mg/ml are similar to that used in the human and animal studies to date.

Monitoring

Daily rectal temperatures were taken and any behavioral/motor changes were noted. Gait monitoring will be conducted as described below based on a four-grade scale for the evaluation of behavioral and motor changes. Grade 0: animal standing, sheep is able to rise and ambulate without any difficulty. Grade 1: Shuffling of either rear leg or slight limp; slight distortion of normal spinal axis. Grade 2: Loss of righting reflex in one of the rear legs, sheep able to stand without assistance, but with some difficulty. Grade 3: Inability to maintain standing posture; attempts to help animal stand are unsuccessful.

Indirect blood pressure, pulse recording, pain testing (mechanical stimulus device, cold and warm water baths see description in procedure) and weight were taken on days 1, 3, 7, 15, 22, 29, 36, and 43. Venous blood samples (15 ml drawn from the jugular vein with Vacutainer brand blood collection tubes) for complete blood count, electrolytes, and extended blood chemistry were drawn on days 1, 15, and 43 (mild procedure only light hand restraint necessary).

On day 43 after venous blood samples were taken, animals were euthanized (Beuthanasia 1 ml/4.5 kg IV bolus injection). CSF samples were obtained for analysis (1-2 ml drawn from L-7/S-1 after a laminectomy was performed to expose the dura) of glucose, total protein, and cell differential.

Animal Preparation and Surgery

Intrathecal catheters and Medtronic infusion pump placements were done in one anesthetic episode under aseptic conditions. Preanesthetic medications consisting of 1 gram of cefazolin and 0.4 mg glycopyrrolate were administered IV prior to induction. Anesthesia was induced with an intravenous bolus cocktail of 0.2 mg/kg diazepam and 6.0 mg/kg ketamine. Animal was intubated with a 8.00 to 10 mm ID cuffed Murphy endotracheal tube. Anesthesia was maintained with halothane or isoflurane at an inspired concentration of approximately 2-3% in oxygen (Ohio ventilator). Distention of the rumen and attendant ventilatory depression were avoided by oral rumen cannulation with a large-bore stomach tube. Body temperature was supported by use of a circulating water pad. Intravenous fluids (0.9% NaCl) were administered throughout the procedure, and vital signs were monitored with an electrocardiogram temperature respirator monitor (Vet/Ox Plus).

After sterile preparation of the surgical field, a midline incision was made over L-6 to S-2 to expose the muscle fascia. A 16-G Tuohy needle was inserted into the intravertebral space at L-7/S-1. The needle was slowly advanced until the dura was punctured and CSF was freely flowing out of the hub of the needle. An intraspinal catheter (4 french ID 0.6 mm×OD 1.2 mm) was threaded into the Tuohy needle and advanced cephalad into the subarachnoid space 10 cm to the approximate level of L-5. The catheter was secured to the muscle fascia with 2-0 silk suture. A pocket was fashioned in the left para lumbar fossa, and the catheter was tunneled to that area with a tunneling device and connected to the pump. The pump was anchored to the muscle in three locations at approximately 90-120 degree intervals with 2-0 silk or 0 braunamid suture. The pump was filled with sterile saline and programmed at the time of surgery to deliver 1 ml/day. The wounds were flushed with a saline/gentamicin solution followed by a local anesthetic. Wounds were closed in layers with vicryl suture. Analgesics (torbugesic 5 mg, IM or morphine up to 10 mg per dose) were administered before sheep emerged from anesthesia and again in the evening and the following morning when the sheep was given antibiotic injections and then as needed thereafter. The postoperative antibiotic regiment consisted of two days of 1 gm cefazolin IM twice daily and then 5 ml Benza-Pen (Penicillin a Benzathine and Penicillin G Procaine) SQ once daily for an additional 3 days.

Although no toxicity or problems were expected from the placement of the spinal catheter and the implanted pump, each animal was observed for any evidence of neurological deficit for 7 days after placement of the catheter and pump.

Pain Testing in the Acute Pain Model

In the sheep model for continuous intrathecal infusion of test substances, testing procedures were developed to determine analgesic activity of an agent.

Mechanical Pain Thresholds

A mechanical stimulus device was used for the pain stimulus. It has a movable blunt pin that supplies pressure to a clipped area in front of the anterior aspect of the radius just above the carpus with increasing force. The pressure from the pin causes the animal to lift its leg, which indicates to the operator to shut the device off. The force applied to the pin was measured by strain gauges that are incorporated into the device on the leg. The output from the strain gauges was recorded on a millivoltmeter in the control box. The mechanical pressure device was fitted to one leg and a dummy device was fitted to the other leg. The sheep was allowed to acclimate to the device. Five baseline test trials were made and averaged (predrug response). To compare the effects of midazolam, the data were accumulated over the testing period, and all response latencies were expressed as percentage of the maximum possible effect (MPE) where:

$$\% MPE = \frac{Postdrug response \text{-} predrug response}{Cutoff \text{-} predrug response} \times 100$$

This test will be conducted prior to starting the sheep being started on drug and again on days 1, 3, 7, 15, 22, 29, 36, 43. The mechanical stimulus device has been developed and validated (Nolan et al. 1987; Kyles et al., 1995) for pain testing in the sheep model.

Thermal Pain Thresholds

Heat. Thresholds to heat stimuli were determined by walking the sheep into a warm water foot bath (maximum temperature not to exceed 55° C.). Prior to drug administration baseline values were recorded by counting the number of times the sheep lifted each leg. This value was then compared to the post drug value on day 1, 7, 15, 22, 29, 36, and 43.

Cold. Thresholds to cold stimuli were determined by walking the sheep into a cold water foot bath (minimum temperature not to fall below 6° C.). Prior to drug administration baseline values were recorded by counting the number of times the sheep lifted each leg. This value was then compared to the post drug value on day 1, 7, 15, 22, 29, 36, and 43.

### EXAMPLE 7

Experimental Procedures Neuropathic Pain Model

Pain Testing in the Neuropathic Pain Model

A mechanical stimulus device as described in Example 6, and/or behavior monitoring as described in Example 5 is used to test analgesic effect in the neuropathic pain model. To compare the effects of midazolam using the mechanical stimulus device, the data were accumulated over the testing period, and all response latencies were expressed as percentage of the maximum possible effect (MPE) where:

$$\% MPE = \frac{Postdrug response \text{-} predrug response}{Cutoff \text{-} predrug response} \times 100$$

This test will be conducted prior to initiation of midazolam infusion and again on days 1, 3, 7, 15, 22, 29, 36, 43 during infusion. The mechanical stimulus device has been developed and validated (Nolan et al. 1987; Kyles et al., 1995) for pain testing in the sheep model.

Surgery

All surgery will be performed under general anesthesia and sterile conditions. Studies are conducted in strict compliance with Guide for the Care and Use of Laboratory Animals and PHS policy on Humane Care and Use of Laboratory Animals.

Preoperative Evaluation

Approximately three days prior to surgery each sheep will undergo a 15-minute baseline behavior evaluation. The observations made in this evaluation include, a computerized behavior software recording (the Observer) for 15minutes—gait monitoring—and recording of pain perception via visual analog scale-using the Observer. In some animals, recording of vital signs (blood pressure, heart rate, respirations per minute) will be performed using the Observer. Baseline values for pain perception are recorded via the mechanical stimulus device and/or behavior monitoring will also be performed at this time.

Gait monitoring will be conducted as described below based on a four-grade scale for the evaluation of behavioral and motor changes. Grade 0: animal standing, sheep is able to rise and ambulate without any difficulty. Grade 1: Shuffling of either rear leg or slight limp; slight distortion of normal spinal axis. Grade 2: Loss of righting reflex in one of the rear legs, sheep able to stand without assistance, but with some difficulty. Grade 3: Inability to maintain standing posture; attempts to help animal stand are unsuccessful.

Surgical Procedures

#1 Sheep instrumented with spinal catheters for probe placement and a subcutaneous spinal infusion pump for drug delivery.

#2 Sheep equipped with arterial and venous subcutaneous femoral ports and the medial, radial, ulnar nerve are ligated (or a combination thereof) to induce neuropathic pain in the animal.

Postoperative Evaluation

The sheep will be observed daily postoperatively. Beginning three days postoperatively (or earlier if the sheep displays neuropathic pain), the sheep will undergo daily observations and behavior tests as previously performed for baseline neuropathic pain measurement. Observations of neuropathic pain development include changes in ambulation, alertness, appetite, urination, defecation, herding activity, body temperature and pain behaviors. This daily behavior is recorded utilizing the observer (an observational software system). If the sheep does not exhibit neuropathic pain it will receive 0.63 mg naloxone. It is possible that animals not exhibiting neuropathic pain may have endogenous opioidergic systems that are tonically activated under pathologically painful conditions, which may inhibit or mask the development of the neuropathic pain. Naloxone will inactivate this endogenous pathway. Once the sheep exhibits neuropathic pain the studies will begin.

Intrathecal Midazolam Efficacy Studies for Neuropathic Pain

Midazolam-niave and nonsteady-state anesthetized studies. The subcutaneous pump filled with saline will be replaced with midazolam (5.0 mg/ml) and programmed to deliver a bolus of 5 mg midazolam, followed by up to 15 mg/day continuous infusion of the desired dose.

Microdialysis sampling on the first day of drug infusion will be performed in some sheep. For this, sheep will be placed under general anesthesia for probe placements in lumbar tissue and lumbar and thoracic CSF. Microdialysis probes will be placed in lumbar CNS tissue percutaneously at L7-S1, two regions of CSF (T10, and L7 spinal levels), and blood to determine midazolam concentration at these regions. Probes will be perfused with an artificial CSF solution at a low flow rate of 2 μL/min. These probes have a 4 mm loop semipermeable membrane at their tip composed of regenerated cellulose (MW cutoff, 18 kD) which allows the passive diffusion of drugs and analytes across a concentration gradient and into the probe effluent. Following placement of microdialysis probes, baseline samples will be collected over one 10 minute interval. After drug infusion has begun, dialysate samples will be continuously collected in 10 minute intervals via a fraction collector for up to three hours.

Once the study is complete probes are removed and the animal will receive 30 mg/kg methylprednisolone sodium succinate IV over a 15-minute period to prevent nervous tissue injury from the temporary probe placement. The animal is then taken off general anesthesia, and allowed to recover. Once recovered, the animal will be placed in an indoor pen and allowed to rest for at least two hours. Following this resting period, the animal will be evaluated for pain relief utilizing the mechanical stimulus device. The sheep will then be returned to the indoor pen and the Observer will be used to evaluate the sheep's behavior.

Midazolam steady-state unanesthetized studies. Microdialysis sampling in awake animals will be performed in some sheep while on therapy. For this, sheep will be placed in a sling inside a movable cart to minimize postural movements. The concentration of midazolam should be at steady state. The same experimental procedure will be followed as explained above, except without the placement of the tissue probes. Samples from probes in CSF will be collected for 3 hours post placement of probes. These microdialysis experiments can be performed once every other week during treatment.

Periodic evaluations of analgesic effect of drug treatment will be performed utilizing the mechanical stimulus device and/or behavior monitoring. The pumps may be programmed to stop the infusion to observe if the animal returns to a neuropathic pain state. Observations and analgesic tests will be performed as for baseline pain assessments once neuropathic pain has returned. Off therapy, the animal will exhibit neuropathic pain usually within 1-3 days. If this pain behavior does not return within 7 days off therapy, the sheep will receive 0.63 mg naloxone. After neuropathic pain is reestablished and recorded, midazolam treatment will be resumed via pump programmed to infuse another bolus of 5 mg midazolam, followed by up to 15 mg/day continuous infusion of the desired dose.

Toxicity Testing of Chronically Administered Intrathecal Midazolam

Animals will be observed daily for any signs of clinical toxicity such as limping and loss of appetite. At necropsy the spinal cord will be examined for any gross changes. Histopathologic evaluation will be performed on all animals after drug studies are complete. These examinations will detect any neurotoxicity that may be present due to the spinal infusion of midazolam.

Pharmacokinetics

Microdialysis methods will be used to determine the steady-state and nonsteady-state pharmacokinetic profile of intrathecally administered midazolam in plasma, CSF, and CNS tissue in the sheep model.

Terminal Tissue Study

On the last day of drug delivery, day 51 post drug initiation (or sooner if neuropathic pain fails to return) a terminal tissue study may be performed. In these studies the sheep will be anesthetized. Microdialysis probes will be placed in three regions of spinal cord tissue and 3 regions of CSF (cisterna magna, T10, and L7 spinal levels), and in venous blood for determination of pharmacokinetic profiles. Partial laminectomies will be performed at these locations to provide adequate visualization of the insertion points and assure proper placement of the probes To place probes in tissue, a small incision is made in the dura and a 16-G introducer is inserted through the incision into the cord. The probe is placed through the introducer and inserted into the cord tissue, the introducer is removed. The probes for CSF sampling are placed through the same incision in the dura. A small introducer is inserted into the incision to elevate the dura while the probe is inserted into the subarachnoid space. The tissue and CSF probes are sealed into place with gel foam and tissue adhesive.

Placement of probes within the cord tissue will be confirmed at the end of each tissue experiment by perfusing the dialysis probes with methylene blue dye for 5 minutes, with subsequent dissection at necropsy by the senior research assistant to assure proper placement.

This model allows for directly sampling from the blood, thoracic, lumbar, and cistemal CSF, and cord tissue. Dialysate samples will be analyzed for midazolam concentration by gas chromatography/mass spectrometry. Pharmacokinetic parameters will be derived by fitting a two or three compartment model to all site-specific drug concentrations. ADAPT II pharmacokinetic software will be used to fit the data. In addition, concentration-time data will be analyzed using a modified signoid Emax model, and will allow generation of a model which integrates measured plasma and local drug concentration data in order to determine “effect compartment” drug concentrations and relate this information to pharmacodynamic outcome (efficacy).

All of the compositions, methods and apparati disclosed and claimed herein can be made and executed without undue experimentation in light of the present disclosure. While the compositions and methods of this invention have been described in terms of preferred embodiments, it will be apparent to those of skill in the art that variations may be applied to the compositions, methods and apparati and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Aguilar et al., Pain, 59:317-320, 1994.

Ahles et al., Pain, 17:277-288, 1983.

Bonica, In: The Management of Pain, Philadelphia, Lea and Febiger (Eds.), 582-585; 410-411, 1990.

Erdine et al., Pain, 80:419-423, 1999.

Gianno et al., In: Intrathecal Drug Therapy for Spasticity and Pain, Springer-Verlag, 1996.

Graham et al., Pain, 8:377-387, 1980.

Hassenbusch et al., J. Neurosurg., 73:405-409,1990.

Jensen et al., Pain, 55:195-203, 1993.

Kremer et al., Pain, 12:153-163, 1982.

Kyles et al. British J. Pharmacology, 108(4):97-913;1995.

Kyles et al., J. Veterinary Pharmacologic. Ther., 18:54-60;1995.

Lesage et al., J. Moffitt Cancer Center, 6(2):136-145, 1999.

Littman et al., Clin. Pharmacol. Ther., 38:16-23, 1985.

Madsen et al., Pain, 5(suppl):S242, 1990.

Malinovsky et al., Anesthesiology, 75:91-97, 1991.

Melzack et al., Pain, 1:277-299, 1975.

Merskey, Pain, 3:S1-225, 1986.

Nishiyama et al., Anesthesiology, 91(2):531-537, 1999.

Nolan et al., J. Pharmacological Methods, 17:39-49;1987.

Schoeffler et al., Reg. Anesthesiology, 16:329-332, 1991.

Schofield et al., Nature, 328:221-227, 1987.

Serrao et al., European J. Anesthesiology, 7:115-122, 1990.

Svensson et al., Reg. Anesthesiology, 20:426-434, 1995.

Valentine et al., European J. Anesthesiology, 13:5589-5593, 1996.

What is claimed is:

1. A method for treating pain in a subject comprising intraspinal administration to said subject of an analgesic formulation comprising preservative-free midazolam, wherein said formulation is substantially free of other analgesic substances.

2. The method of claim 1, wherein the treatment is for neuropathic pain.

3. The method of claim 1, wherein the treatment is for non-neuropathic pain.

4. The method of claim 1, wherein midazolam is provided at high doses.

5. The method of claim 4, wherein the daily dose of midazolam is at least about 1.0 mg.

6. The method of claim 5, wherein the daily dose of midazolam is at least about 5.0 mg.

7. The method of claim 6, wherein the daily dose of midazolam is at least about 10.0 mg.

8. The method of claim 7, wherein the daily dose of midazolam is at least about 15.0 mg.

9. The method of claim 1, wherein said formulation is administered gradually over a time period of greater than one minute.

10. The method of claim 1, wherein said formulation is administered gradually over a time period of greater than ten minutes.

11. The method of claim 1, wherein said formulation is administered gradually over a time period of greater than thirty minutes.

12. The method of claim 1, wherein said formulation is administered gradually over a time period of greater than sixty minutes.

13. The method of claim 1, wherein said formulation is administered gradually over a time period of greater than one-hundred twenty minutes.

14. The method of claim 1, wherein said formulation is administered gradually over a time period of greater than four hours.

15. The method of claim 1, wherein said formulation is administered gradually over a time period of greater than eight hours.

16. The method of claim 1, wherein said formulation is administered gradually over a time period of greater than twelve eight hours.

17. The method of claim 1, wherein said formulation is administered gradually over a time period of greater than twenty-four hours.

18. The method of claim 9, wherein said formulation is administered by a continuous infusion pump.

19. The method of claim 18, wherein said pump is implanted subcutaneously in said subject.

20. The method of claim 1, wherein said subject has cancer.

21. The method of claim 20, wherein said subject has cancer pain.

22. The method of claim 20, wherein the cancer pain is a neuropathic pain.

23. The method of claim 20, wherein the cancer pain is a non-neuropathic pain.

24. The method of claim 1, wherein said subject is opioid tolerant.

25. The method of claim 1, wherein said subject suffers from opioid-resistant neuropathic pain.

26. The method of claim 1, wherein said subject is a human.

27. The method of claim 1, wherein said analgesic formulation comprises midazolam at about 2.5 to about 5.0 mg/ml.

28. The method of claim 1, wherein toxicity is measured during treatment.

29. The method of claim 28, wherein a dose modification is made based on said toxicity measurement.

30. The method of claim 1, wherein pain relief is measured during treatment.

31. The method of claim 30, wherein a dose modification is made based on said pain relief measurement.
